(19) United States
(12) Patent Application Publication
(10) Pub. No.: US 2018/0050035 A1
(43) Pub. Date: Feb. 22, 2018

(54) ANTIFUNGAL TREATMENT OF ROSACEA

(71) Applicant: Gerbe Labs Inc., Los Altos, CA (US)

(72) Inventor: Ronald W. Gerbe, Chapel Hill, NC (US)

(21) Appl. No.: 15/682,495

(22) Filed: Aug. 21, 2017

Related U.S. Application Data
(60) Provisional application No. 62/495,606, filed on Jan. 19, 2017.
(60) Provisional application No. 62/428,884, filed on Dec. 1, 2016.
(60) Provisional application No. 62/377,355, filed on Aug. 19, 2016.

(51) Int. Cl. A61K 31/496; A61K 45/06; A61K 31/7036; A61K 38/14; A61K 31/58; A61K 9/00

(52) U.S. Cl. CPC A61K 31/496; A61K 45/06; A61K 9/0014; A61K 38/14; A61K 31/58; A61K 31/7036

(57) ABSTRACT

Methods of treating disorders with a combination of antifungals is provided. The methods include topically administering a combination of antifungal agents to an affected tissue are provided. For example, the ostiomeatal complex of a subject having a recurring condition can receive the formulations taught herein, the topically administering including contacting a first antifungal agent with the ostiomeatal complex, the first antifungal selected as effective at killing Candida species of fungus, inhibiting the growth and/or reproduction of the Candida species, or a combination thereof; and, contacting a second antifungal agent with the ostiomeatal complex, the second antifungal selected as effective at killing an Aspergillus species of fungus, inhibiting the growth and/or reproduction of the Aspergillus species, or a combination thereof; wherein, the topically administering of the first antifungal agent is concurrent with the topically administering of the second antifungal agent.

### CROSS-REFERENCE TO RELATED APPLICATIONS

This application claims the benefit of U.S. Provisional Application Nos. 62/495,606, filed Jan. 19, 2017, 62/428,884, filed Dec. 1, 2016, and 62/377,355, filed Aug. 19, 2016; wherein, each application of which is hereby incorporated herein by reference in its entirety.

### BACKGROUND

### Field of the Invention

The teachings provided herein generally relate to the administration of a combination of antifungal agents to a subject to treat a variety of disorders.

### Description of Related Art

One of skill will appreciate that there are several health conditions that either have no treatment, or have a treatment that is less than desirable. This teaching addresses conditions that have been found to be treatable by a combination of antifungal agents, thereby either finally providing an adequate treatment or otherwise providing an improvement over treatments currently available.

Chronic rhinosinusitis (CRS) is one of these problematic conditions, as it still presents a clinically significant technical problem. Unfortunately, this significant problem is a common condition in which the sinuses become inflamed and swollen for at least 12 weeks, despite treatment attempts, and it remains in need of an effective treatment. It interferes with sinus drainage and causes mucus buildup, such that breathing through your nose becomes difficult, your eyes and face can feel swollen with facial pain or tenderness, effecting productivity and quality of life significantly. Daily saline irrigation with topical cortical steroid therapy appears to be the most commonly used therapy for chronic rhinosinusitis as an attempt to reduce mucosal edema, promote sinus drainage. To eradicate infections, oral antibiotics are also often used, but the role of bacteria in the pathogenesis of chronic rhinosinusitis is still considered to be debatable in the art. Sometimes, an early diagnosis and intensive treatment with oral antibiotics, topical nasal steroids, decongestants, and saline nasal sprays results in symptom relief in a significant number of patients, and sometimes the condition can be cured. If the treatments fail, however, meaning there is a repeated recurrence of the condition, the patient may need to suffer sinus surgery. In fact, See, for example, http://emedicine.medscape.com/article/232791-treatment (Updated Apr. 21, 2017; downloaded May 8, 2017).

Rheumatoid arthritis is another such condition that can be treated using the methods taught herein. Rheumatoid arthritis is a long-term autoimmune disorder that primarily affects joints. It typically results in warm, swollen, and painful joints, and the pain and stiffness often worsen following rest. Most commonly, the wrist and hands are involved, with the same joints typically involved on both sides of the body.

Crohn's disease is another such condition that can be treated using the methods taught herein. is a condition that be treated using the methods taught herein. Crohn's disease is a type of inflammatory bowel disease (IBD) that may affect any part of the gastrointestinal tract from mouth to anus. Signs and symptoms often include abdominal pain, diarrhea (which may be bloody if inflammation is severe), fever, and weight loss. Other complications may occur outside the gastrointestinal tract and include anemia, skin rashes, arthritis, inflammation of the eye, and feeling tired. The skin rashes may be due to infections as well as pyoderma gangrenosum or erythema nodosum. Bowel obstruction also commonly occurs and those with the disease are at greater risk of bowel cancer. It results in a chronic inflammatory disorder, in which the body's immune system attacks the gastrointestinal tract possibly directed at microbial antigens.

Rosacea is another such condition that can be treated using the methods taught herein. Rosacea is a long-term skin condition that typically affects the face. It results in redness, pimples, swelling, and small and superficial dilated blood vessels. Often the nose, cheeks, and forehead, and chin are most involved. A red enlarged nose may occur in severe disease, a condition known as rhinophyma. Unfortunately, the cause of Rosacea is considered to be unknown to those skilled in the art and, as such, the condition is considered to be incurable at this time. Topical treatments are used to treat symptoms using, for example, metronidazole, doxycycline, or tetracycline. Other treatments that are sometimes used, and may show a benefit, include, for example, brimonidine cream, ivermectin cream, and isotretinoin; and, dermabrasion or laser surgery may also be used.

Psoriasis is a condition that be treated using the methods taught herein. Psoriasis is considered to be a long-lasting autoimmune disease which is characterized by patches of abnormal skin, and these skin patches are typically red, itchy, and scaly, varying in severity from small and localized to complete body coverage. The main types of psoriasis can be treated using the methods provided herein and include plaque, guttate, inverse, pustular, and erythrodermic psoriasis. Plaque psoriasis, also known as psoriasis vulgaris, makes up about 90% of all cases and typically presents with red patches with white scales on top. Areas of the body most commonly affected are the back of the forearms, shins, around the navel, and the scalp. Guttate psoriasis has drop-shaped lesions. Pustular psoriasis presents with small non-infectious pus-filled blisters. Inverse psoriasis forms red patches in skin folds, and erythrodermic psoriasis occurs when the rash becomes very widespread, and can develop from any of the other types. One of skill in the art will appreciate that there is currently no cure for psoriasis. However, various treatments can help control the symptoms, and these treatments may include steroid creams, vitamin D3 cream, ultraviolet light, and immune system suppressing medications such as methotrexate. About 75% of cases can be managed with creams alone, and the disease typically affects 2-4% of the population.

Cardiovascular disease is a condition that be treated using the methods taught herein. Cardiovascular disease involves the heart or blood vessels and includes coronary artery diseases (CAD) such as angina and myocardial infarction (commonly known as a heart attack). CVDs include stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis.

Accordingly, and for at least the above reasons, those of skill in the art will appreciate having highly effective methods of treating a variety of conditions that otherwise have no method of treatment, or perhaps only inadequate methods of treatment, by treating a fungal infection, for example, an infection by Candida and/or Aspergillus species of fungus and, particularly, providing effective methods of alleviating symptoms and inhibiting and/or stopping the recurrence of such conditions by treating the fungal source of the condtion(s). For example, such methods can treat (i) inflammation that ulcerates mucosal lining of the ostiomeatal complex and (ii) secondary infections that can result from ulceration of the mucosal lining, alleviating conditions that are concurrent with such fungal infections, while (iii) lowering the total amount of antifungal required for administration, lowering the toxic effect and (iv) reducing the possibility of a recurrence of the condition in the subject.

### SUMMARY

The teachings provided herein generally provide a treatment of disorders using an antifungal combination. In some embodiments, the teachings are directed to methods of treating a fungal infection. The methods can comprise administering a combination of antifungal agents to a tissue infected with one or more fungi. For example, the fungi can include a combination of a Candida species of fungus and an Aspergillus species of fungus. The administering can comprise a topical administration. In some embodiments, the topical administration can include, for example, contacting the tissue with a first antifungal agent, the first antifungal agent selected as effective at killing the Candida species, inhibiting the growth and/or reproduction of the Candida species, or a combination thereof; and, contacting the tissue with a second antifungal agent, the second antifungal agent selected as effective at killing the Aspergillus species, inhibiting the growth and/or reproduction of the Aspergillus species, or a combination thereof; wherein, the topically administering of the first antifungal agent is concurrent with the topically administering of the second antifungal agent.

In some embodiments, the tissue includes the ostiomeatal complex. And, in some embodiments, the method is a treatment for rheumatoid arthritis. In some embodiments, the method is a treatment for Crohn's disease. In some embodiments, the method is a treatment for rosacea. In some embodiments, the method is a treatment for cardiomyopathy. In some embodiments, the method is a treatment for a condition selected from the group consisting of chronic rhinosinusitis, colitis, psoriasis, ankylosing spondylitis, Addison's disease, cardiomyopathy, celiac disease, Graves' disease, lupus, Meniere's disease, multiple sclerosis, myositis, and scleroderma.

In some embodiments, the method is an administration of a combination of antifungals that can include an imidazole; and, in some embodiments, the imidazole is itraconazole. In some embodiments, the method is an administration of a combination of antifungals that can include an triazole; and, in some embodiments, the triazole is ketoconazole. In some embodiments, the combination of antifungals includes an imidazole and a triazole; and in some embodiments, the combination of antifungals includes itraconazole and ketoconazole.

In some embodiments, the methods include further administering an antibiotic to the subject. In some embodiments, the antibiotic is an aminoglycoside; and, in some embodiments, the aminoglycoside is tobramycin. In some embodiments, the antibiotic is a glycopeptide; and, in some embodiments, the glycopeptide is vancomycin.

In some embodiments, the methods further include administering a corticosteroid to the subject; and, in some embodiments, the corticosteroid is a Group III or Group IV corticosteroid selected from the group consisting of triamcinolone acetonide, mometasone furoate, fluticasone propionate, betamethasone dipropionate, halometasone, fluocinolone acetonide, hydrocortisone valerate, hydrocortisone butyrate, flurandrenolide, and triamcinolone acetonide. And, in some embodiments, the corticosteroid is mometasone furoate.

In some embodiments, the combination of the antifungal agents are administered concurrently as a formulation with a Group III or Group IV corticosteroid selected from the group consisting of triamcinolone acetonide, mometasone furoate, fluticasone propionate, betamethasone dipropionate, halometasone, fluocinolone acetonide, hydrocortisone valerate, hydrocortisone butyrate, flurandrenolide, and triamcinolone acetonide; and, an antibiotic selected from the group consisting of an aminoglycoside and a glycopeptide; wherein, the administering is repeated daily for at least 3 weeks.

In some embodiments, the methods further include dilating sinus ostia, and the administering is repeated daily for at least 3 weeks. And, in some embodiments, the methods further include dilating sinus ostia; and, the combination of the antifungal agents include itraconazole and ketoconazole. In these embodiments, the antifungal agents can be administered concurrently as a formulation that includes a corticosteroid that includes mometasone furoate; and, an antibiotic that includes tobramycin and/or vancomycin; wherein, the administering is repeated daily for at least 3 weeks, and then once a day for at least 1 additional month.

In some embodiments, a formulation used in the methods is provided and comprises a first antifungal selected as effective at killing Candida species of fungus, inhibiting the growth and/or reproduction of the Candida species, or a combination thereof; and, a second antifungal selected as effective at killing an Aspergillus species of fungus, inhibiting the growth and/or reproduction of the Aspergillus species, or a combination thereof. In some embodiments, the first antifungal includes itraconazole; the second antifungal includes ketoconazole; and, the formulation further comprises a corticosteroid that includes mometasone furoate; and, an antibiotic that includes tobramycin and/or vancomycin; wherein, the formulation at least substantially inhibits the recurrence of the condition treated in a group of subjects having the condition and receiving an administration of the formulation to each of their respective ostiomeatal complexes when compared to a group of subjects having the condition and not receiving an administration of the formulation.

### BRIEF DESCRIPTION OF THE FIGURES

FIGS. 1A-1C illustrate healthy sinuses, sinuses with chronic sinusitis, and an ostiomeatal complex of a human, according to some embodiments.

FIGS. 2A-2D illustrates a prior art balloon sinuplasty procedure that opens the ostiomeatal complex of a human, according to some embodiments.

FIG. 3 illustrates a method of treating a chronic rhinosinusitis, according to some embodiments.

FIGS. 4A and 4B illustrate the components of an assay tray, and the assay tray, used in an MIC assay to identify component synergy in embodiments taught herein, according to some embodiments.

FIG. 5 shows the synergy data for Itraconazole and Ketoconazole against Aspergillus fumigatus, according to some embodiments.

FIG. 6 shows the synergy data for Itraconazole and Ketoconazole against Candida albicans, according to some embodiments.

FIG. 7 shows the synergy data Itraconazole and Ketoconazole against Aspergillus fumigatus+Candida albicans, according to some embodiments.

FIGS. 8A and 8B illustrate the use of the methods taught herein in the treatment of rheumatoid arthritis, according to some embodiments.

FIGS. 9A and 9B illustrate the use of the methods taught herein in the treatment of Crohn's disease, according to some embodiments.

FIGS. 10A and 10B illustrate the use of the methods taught herein in the treatment of rosacea, according to some embodiments.

FIGS. 11A and 11B illustrate the use of the methods taught herein in the treatment of psoriasis, according to some embodiments.

FIGS. 12A and 12B illustrate the use of the methods taught herein in the treatment of cardiovascular disease, according to some embodiments.

### DETAILED DESCRIPTION

The teachings provided herein generally provide a treatment of disorders using an antifungal combination. In some embodiments, the teachings are directed to methods of treating a fungal infection. The methods can comprise administering a combination of antifungal agents to a tissue infected with one or more fungi. For example, the fungi can include a combination of a Candida species of fungus and an Aspergillus species of fungus. The administering can comprise a topical administration. In some embodiments, the topical administration can include, for example, contacting the tissue with a first antifungal agent, the first antifungal agent selected as effective at killing the Candida species, inhibiting the growth and/or reproduction of the Candida species, or a combination thereof; and, contacting the tissue with a second antifungal agent, the second antifungal agent selected as effective at killing the Aspergillus species, inhibiting the growth and/or reproduction of the Aspergillus species, or a combination thereof; wherein, the topically administering of the first antifungal agent is concurrent with the topically administering of the second antifungal agent.

The administration of the treatments provided herein includes a topical administration of any agent or formulation of agents to the ostiomeatal complex of a subject. In some embodiments, the administration can include a balloon dilation, for example, which can include the use of a balloon sinuplasty procedure. In some embodiments, the administration includes an irrigation, such as an irrigation of the ostiomeatal complex of a subject. In some embodiments, the administration includes the use of a nebulizer, such as a nebulization of the ostiomeatal complex of a subject.

The terms “treat,” “treating,” and “treatment” can be used interchangeably in some embodiments and refer to the administering or application of the compositions and formulations taught herein, and all administrations directed to the prevention, prophylaxis, inhibition, amelioration of the symptoms, or even a cure of a condition taught herein. The terms “disease,” “condition,” “disorder,” and “ailment” can be used interchangeably in some embodiments. The term “subject” and “patient” can be used interchangeably in some embodiments and refer to an animal such as a mammal including, but not limited to, non-primates such as, for example, a cow, pig, horse, cat, dog, rat and mouse; and primates such as, for example, a monkey or a human. As such, the terms “subject” and “patient” can also be applied to non-human biologic applications including, but not limited to, veterinary, companion animals, commercial livestock, and the like.

The compositions provided herein can be referred to as compositions, compounds, agents, active agents, bioactive agents, supplements, drugs, and the like. In some embodiments, the terms “composition,” “compound,” “agent,” “active”, “active agent”, “bioactive agent,” “supplement,” and “drug” can be used interchangeably and, it should be appreciated that, a “formulation” can comprise any one or any combination of these. Likewise, in some embodiments, the composition can also be in a liquid or dry form, where a dry form can be a powder form in some embodiments, and a liquid form can include an aqueous or non-aqueous component. Moreover, the term “bioactivity” can refer to the function of the compound when administered orally, topically, or perhaps rectally to a subject.

In some embodiments, the term “target site” can be used to refer to a select location on or in a subject that could benefit from an administration of a compound taught herein. As such, the teachings include a method of administering one or more compounds taught herein to a healthy or damaged tissue. In some embodiments, for example, the target site is the ostiomeatal complex of a subject.

In some embodiments, the treatments can be administered in the same way for any of the conditions treated. For example, in some embodiments, an administration of a formulation provided herein can target the ostiomeatal region of the subject being treated, whether the condition treated is chronic rhinosinusitis, rheumatoid arthritis, Crohn's disease, rosacea, psoriasis, or any other condition set-forth herein. One of skill will appreciate, however, that the administrations can differ in some embodiments, for example, with respect to the type and amount of antifungals delivered, the type and amount of antibiotic delivered, the type and amount of anti-inflammatory delivered, and the target tissue selected for delivery, as well as the course and duration of treatment of the subject.

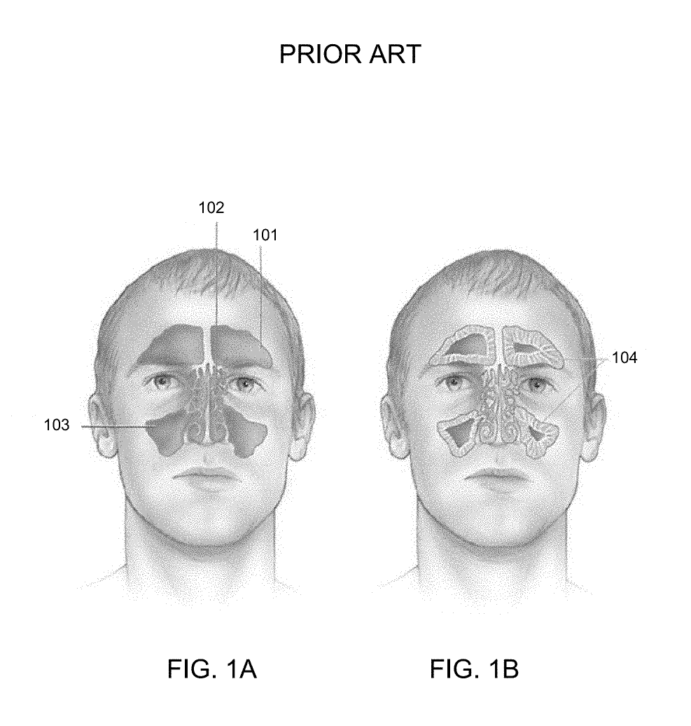

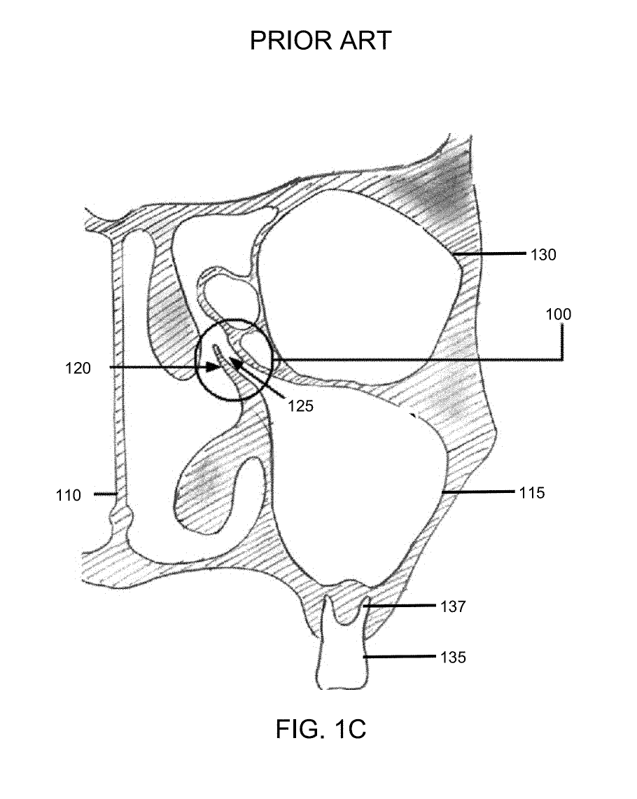

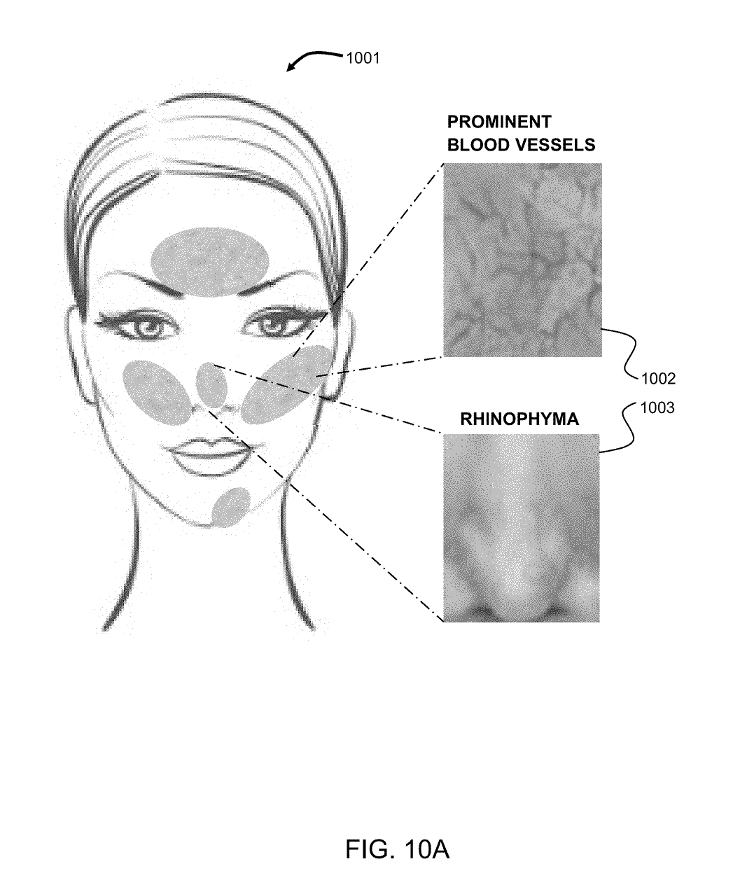

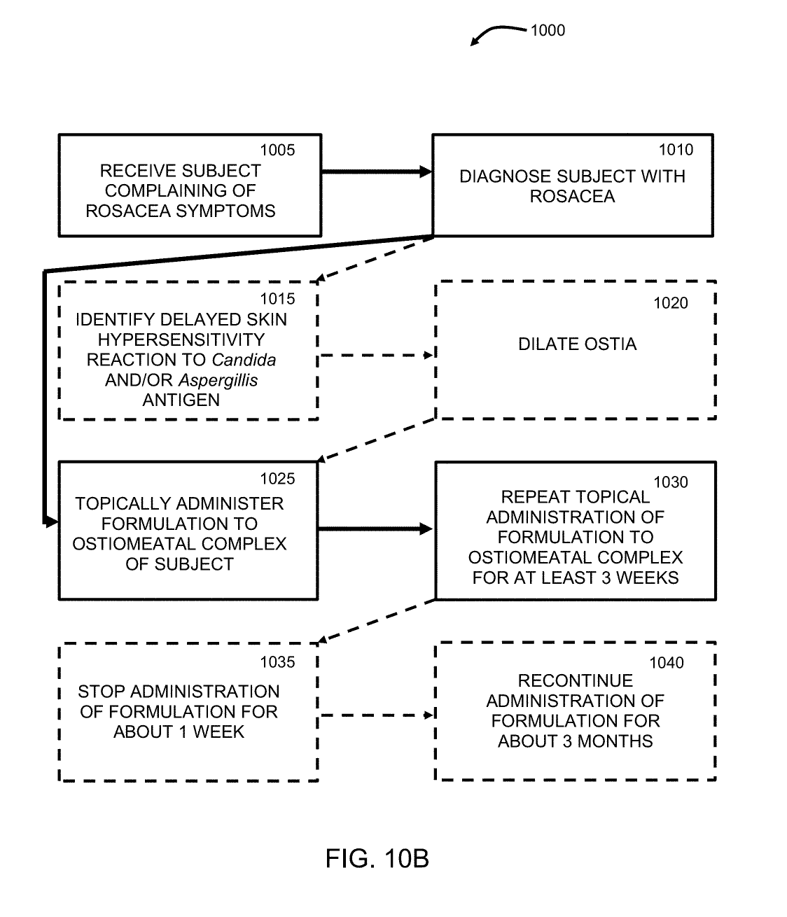

FIGS. 1A-1C illustrate healthy sinuses, sinuses with chronic sinusitis, and an ostiomeatal complex of a human, according to some embodiments. As illustrated in FIG. 1A, there are four paired (paranasal) sinuses in the human skull: the frontal sinuses 101, the ethmoid sinuses 102, the maxillary sinuses 103, and the sphenoid sinus (not shown, but posterior to ethmoid sinuses in the sphenoid bone), each of which is named for the region of the skull for which they inhabit. The paranasal sinuses are lined with ciliated respiratory epithelium and this epithelium is innervated and vascular. Mucus is moved by the ciliary component of the respiratory epithelium to the ostia or “windows” by which these paranasal sinuses communicate with the nasal cavity. These ostia interact and communicate with the nasal cavity through the ostiomeatal complex. Healthy sinuses, as shown in FIG. 1A, are filled with air, whereas sinusitis, is shown in FIG. 1B, and is an inflammation and swelling 104 of the tissue lining the sinuses. Inflammation is the common theme in chronic sinusitis, and current therapies are lacking effectiveness in treating of this uncomfortable and often painful condition that significantly affects quality of life and often is tied to conditions that are more severe. In this teaching, the importance of the ostiomeatal complex 100 as a target tissue in the treatment of chronic sinusitis is emphasized and shown in FIG. 10. FIG. 10 shows structures present around the ostiomeatal complex 100, including the septum 110, the maxillary sinus 115, the uncinated process 120, and the infundibulum 125, which is a passageway that communicates with the maxillary sinus 115. The orbit 130 is also shown in the image and is closely positioned to the ostiomeatal complex 100. Also shown is a tooth 135 with roots 137 in very close proximity to the border of the maxillary sinus 115. The proximity of the orbit 130, tooth 135 and root 137 illustrate why maxillary sinus pressure can result in referred pain to the teeth and eye.

One of skill will appreciate that the signs and symptoms of rhinosinusitis, in general, can be divided into “major” and “minor” categories. Major signs and symptoms include, for example, facial pain/pressure/fullness; nasal obstruction/blockage; nasal or postnasal discharge/purulence (by history or physical examination); hyposmia/anosmia; and fever (in acute rhinosinusitis only). Minor signs and symptoms include, for example, headaches; fever (other than acute rhinosinusitis); halitosis; fatigue; dental pain; cough; and ear pain/pressure/fullness. The presence of rhinosinusitis is based on observing two or more major signs and symptoms; one major and two or more minor signs or symptoms; or, nasal purulence on examination. There are 4 main groups of rhinosinusitis: acute (persists up to 4 weeks), subacute (persists from 4 weeks to <12 weeks), recurrent acute (4 or more episodes per year of acute rhinosinusitis, each at least 7 days long with no signs and symptoms between episodes), and chronic (persists for 12 weeks or more). See, for example, http://www.aafp.org/afp/2001/0101/p69.html.

Once chronic rhinosinusitis has been properly identified for treatment, one or methods of treatment can then be selected. The American Academy of Otolaryngology-Head and Neck Surgery Foundation has recently updated its clinical practice guidelines for such an analysis in the identification and treatment of chronic rhinosinusitis:
Distinguish acute bacterial rhinosinusitis from acute rhinosinusitis caused by viral upper respiratory infections and noninfectious conditions;
Confirm the clinical diagnosis of CRS with objective documentation of sinonasal inflammation via anterior rhinoscopy, nasal endoscopy, or CT scan;
Offer either watchful waiting (without antibiotics) or initial antibiotic therapy for adults with uncomplicated acute bacterial rhinosinusitis;
If antibiotics are prescribed, the initial antibiotic therapy for adults with uncomplicated acute bacterial rhinosinusitis should be amoxicillin with or without clavulanate as first-line therapy for 5-10 days;
Reassess the patient to confirm to bacterial rhinosinusitis and exclude other causes of illness; assess for complications if the patient worsens or fails to improve with the initial management option by 7 days after diagnosis or worsens during the initial management;
Distinguish CRS and recurrent acute rhinosinusitis from isolated episodes of acute bacterial rhinosinusitis and other causes of sinonasal symptoms;
Assess patients with CRS or recurrent acute rhinosinusitis for multiple chronic conditions that would modify management (eg, asthma, cystic fibrosis, immunodeficiency, ciliary dyskinesia);
Assess for nasal polyps in patients with CRS;
Recommend saline nasal irrigation, topical intranasal corticosteroids, or both for symptomatic relief of CRS;
Do not prescribe topical or systemic antifuncial therapy in patients with CRS;
   See, for example, Rosenfeld R. M., et al. Clinical practice guideline (update): adult sinusitis. Otolaryngol Head Neck Surg. 152 (2 Suppl):S1-S39 (April, 2015); see also, Brook ltzhak, et al. Chronic rhinosinusitis Treatment & Management. http://emedicine.medscape.com/article/232791-treatment (Updated Apr. 21, 2017; downloaded May 8, 2017).

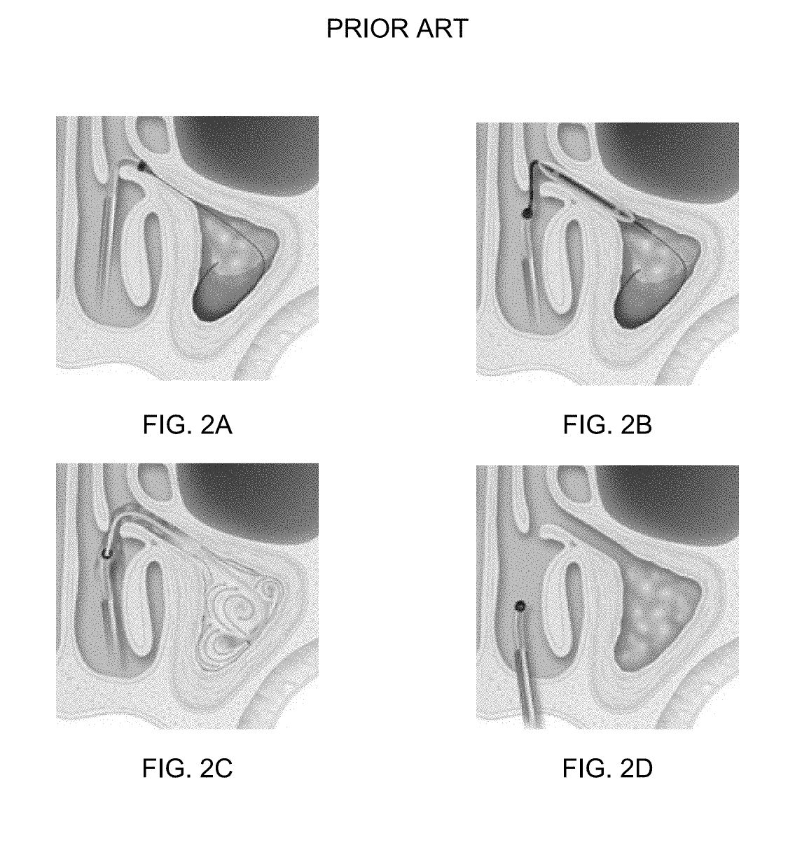

FIGS. 2A-2D illustrate a balloon sinuplasty procedure, according to some embodiments. Balloon sinuplasty is a procedure that dilates the ostia, such that it opens inflamed sinuses similar to how a heart surgeon opens blocked arteries with balloon angioplasty. The procedure is effective at relieving symptoms of chronic rhinosinusitis by opening the blocked sinus passageways and drain mucus build-up. In FIG. 2A a balloon catheter system is inserted in the inflamed maxillary sinus, and the balloon is inflated in FIG. 2B to expand the sinus opening known as the ethmoidal infundibulum. Saline is sprayed into the maxillary sinus to flush out pus and mucus in FIG. 2C, and the system is removed in FIG. 2D to leave the sinuses open.

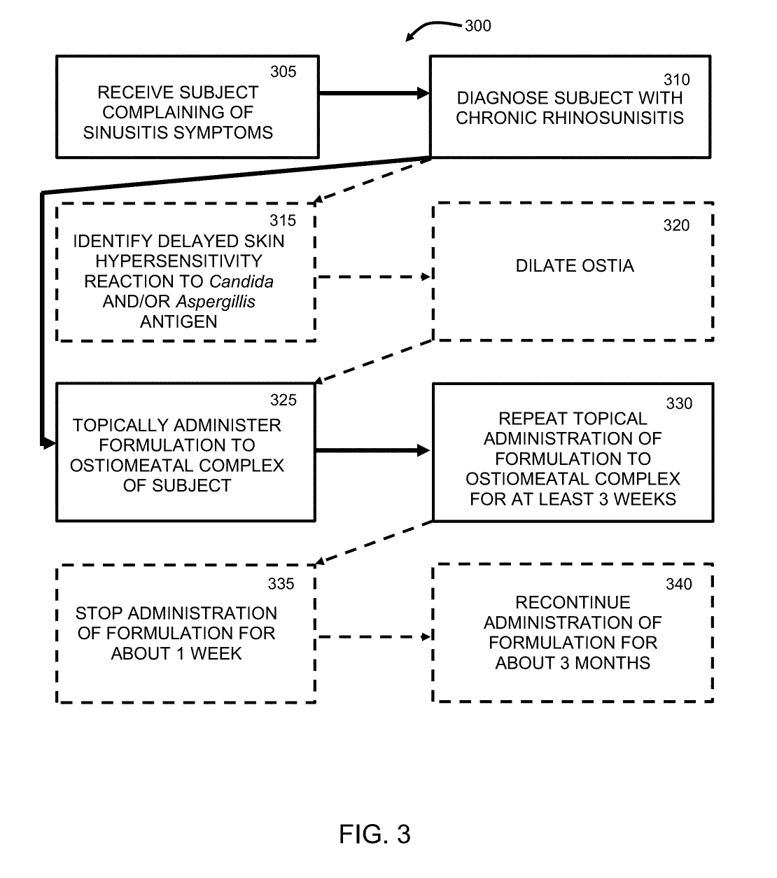

FIG. 3 illustrates a method of treating a chronic rhinosinusitis, according to some embodiments. The method 300 of FIG. 3 generally relates to treatment of chronic rhinosinusitis, although it can extend to treatments of other conditions that involve an autoimmune response through the administration of the formulations taught herein to human secretory tissue. In some embodiments, in the treatment of chronic rhinosinusitis, the methods can include first receiving 305 a subject that is complaining of sinusitis symptoms and then diagnose 310 the subject with chronic rhinosinusitis through an accepted analysis of the symptoms before initiating any treatment set-forth herein.

A topical administration of the combination of the antifungal agents can then be repeated 330 and, in some embodiments, repeated for at least 3 weeks. In these embodiments, the topically administering can include contacting a first antifungal agent with the ostiomeatal complex, the first antifungal selected as effective at killing Candida species of fungus, inhibiting the growth and/or reproduction of the Candida species, or a combination thereof; and, contacting a second antifungal agent with the ostiomeatal complex, the second antifungal selected as effective at killing an Aspergillus species of fungus, inhibiting the growth and/or reproduction of the Aspergillus species, or a combination thereof. In such embodiments, the topically administering of the first antifungal agent can be concurrent with the topically administering of the second antifungal agent. It should be appreciated that the agents, compositions, and formulations taught herein are administered to effectively kill the survival spores of each type of fungus taught herein in order to at least inhibit, and preferably prevent, the recurrence of the fungus, and thus the chronic sinusitis, in the subject being treated.

In some embodiments, the method can further comprise topical administration of any one or any combination of corticosteroids to the subject. In some embodiments, the method can further comprise topical administration of any one or any combination of antibiotics to the subject. In some embodiments, the method can further comprise topical administration of any one or any combination of antibiotics and any one or any combination of corticosteroids to the subject.

In some embodiments, the combination of the antifungal agents can be administered concurrently as a formulation. In some embodiments, the combination of antifungal agents can be administered in formulation with a corticosteroid and/or an antibiotic. In other words, in some embodiments, each of the antifungals can be administered separately or each administered as a component of the same formulation. Likewise, in some embodiments, the antibiotic can be administered separate from at least one of the antifungals and, in some embodiments, the antibiotic is administered with the combination of antifungals in the same formulation. Likewise, in some embodiments, the corticosteroid can be administered separate from at least one of the antifungals and, in some embodiments, the steroid can be administered with the combination of antifungals in the same formulation. It should be appreciated that the combination of antifungals, the antibiotic, and the steroid, can be administered together in the same formulation.

In some embodiments, the administering of the combination of antifungals can be repeated daily for at least 3 weeks. And, in some embodiments, the administering further comprises dilating sinus ostia prior to administration of an agent or formulation taught herein, and the administering is repeated daily for at least 3 weeks. In some embodiments, the administering is repeated daily for at least 3 weeks, and then once a day for at least 1 additional month.

It should be appreciated that in the embodiments taught herein, the formulation is effective in at least substantially inhibiting the recurrence of chronic rhinosinusitis in a group of subjects having chronic recurring sinusitis and receiving an administration of the formulation to each of their respective ostiomeatal complexes when compared to a group of subjects having chronic recurring sinusitis and not receiving an administration of the formulation.

In some embodiments, a skin test might also be used to identify 315 a delayed skin hypersensitivity reaction to an inhaled fungal antigen. For example, a positive delayed inflammatory reaction to an inhaled antigen in the subject may include a Candida species of fungi and/or an Aspergillus species of fungi. In some embodiments, such a reaction may include Candida species selected from the group consisting of Candida albicans, Candida tropicalis, Candida glabrata, and Candida parapsilosis. Likewise, in some embodiments, such a reaction may include Aspergillus species selected from the group consisting of Aspergillus fumigatus, Aspergillus nigra (niger), and Aspergillus flavus. Other species of fungi may be identified as causing a similar reaction to skin testing and treated according to the methods taught herein including, for example, Alternia species and Cladosporium species, namely Cladosporium cladosporioides. Most cases of chronic sinusitis are inflammatory with only a questionable relationship to traditional bacterial infectious etiologies. However, it should be appreciated that acute exacerbations of chronic rhinosinusitis are commonly seen in patients who have already had one or many prior sinus surgeries. The organisms cultured in these patients can include Staphylococcus aureus, Pseudomonas aeruginosa, and acute pathogens such as Streptococcus pneumoniae and Haemophilus influenza. See, for example, Ferguson, Berrylin J. Chronic rhinosinusitis. http://www.antimicrobe.org/e15.asp (downloaded May 18, 2017).

In some embodiments, dilation 320 of the ostia can be included in the method to expand the sinus openings and allow the agents and formulations taught herein to better reach the ostiomeatal complex. A balloon dilation procedure is discussed above with reference to FIGS. 2A-2D. The goal of the procedure includes atraumatically opening the ostia of the sinuses to facilitate drainage and topical treatment of the sinuses by a micro-reduction of the inflamed sinus tissue. The dilation facilitates the evacuation of encapsulated fungal spores as well as exposing additional surface area in the ostiomeatal complex for enhancing delivery of the compositions and formulations taught herein.

In some embodiments, after the topical administration is repeated 330, the treatment can stop for about a week or so, and then the administration can be re-continued 340 for about 3 more months or so in an effort to eliminate the fungal survival spores responsible for triggering the inflammatory reaction essential in recurrent sinusitis.

As such, the teachings are, of course, also directed to formulations comprising a first antifungal selected as effective at killing Candida species of fungus, inhibiting the growth and/or reproduction of the Candida species, or a combination thereof; and, a second antifungal selected as effective at killing an Aspergillus species of fungus, inhibiting the growth and/or reproduction of the Aspergillus species, or a combination thereof. In some embodiments, the second antifungal is selected as effective at killing both Aspergillus species and Candida species of fungi, inhibiting the growth and/or reproduction of both species, or a combination thereof.

In some embodiments, the combination of antifungals includes an imidazole. And, in some embodiments, the imidazole is itraconazole. In some embodiments, the combination of antifungals includes a triazole. And, in some embodiments, the triazole is ketoconazole. As such, in many embodiments, the combination of antifungals will include an imidazole and a triazole. In some embodiments, the combination of antifungals includes itraconazole and ketoconazole.

The antibiotics can be selected based on what is known about the presence of an infection in the subject. In some embodiments, the antibiotic includes an aminoglycoside. And, in some embodiments, the aminoglycoside is tobramycin. In some embodiments, the antibiotic is a glycopeptide. And, in some embodiments, the glycopeptide is vancomycin, for example, in subjects having a Staphylococcus infection, such as a Staphylococcus aureus infection, and particularly in subjects having a methicillin-resistant Staphylococcus aureus (MRSA) infection.

In some embodiments, the corticosteroid can include any one or combination of Group I, Group II, Group II, Group IV, Group V, Group VI, and/or Group VII corticosteroids. In some embodiments, the corticosteroid is a Group III or Group IV corticosteroid selected from the group consisting of triamcinolone acetonide, mometasone furoate, fluticasone propionate, betamethasone dipropionate, halometasone, fluocinolone acetonide, hydrocortisone valerate, hydrocortisone butyrate, flurandrenolide, and triamcinolone acetonide. As such, in some embodiments, the corticosteroid can include mometasone furoate.

### Methods of Use

It should appreciated that, in addition to the treatments taught above, the agents taught herein can be used for any of a variety of other treatments, preventative, prophylactic, ameliorative, or otherwise. The uses can include treatment of an existing condition. In some embodiments, any target tissue that can make contact with one or more active components taught herein can be treated. In some embodiments, a tissue can have a desirable secondary effect from one or more of the active components taught herein making contact elsewhere in the subject, such that one or more of the active components can contact a first tissue, the target tissue, whereas a second tissue realizes a beneficial effect. For example, the first tissue can be an ostiomeatal complex, and the second tissue can realize the desirable effect. In some embodiments, the desirable effect can include a release of a neurotransmitter or a neuroimpulse. The tissue that is the target, or perhaps the secondary tissue, can be any tissue, for example, human secretory tissue, connective, muscle, nervous, and/or epithelial tissue. In some embodiments, the tissue is a human secretory tissue. In some embodiments, the tissue is a mucosal tissue. And, in some embodiments, the tissue is gastrointestinal tissue.

Secretory granules in the human body share a common antigenic signature. The term “secretory tissue” can include, but is not limited to, the sinus mucous membrane, secretory glands of the paranasal sinuses; the pancreas; serotonin glands in the brain; the intima (inner lining) of the arterial circulation, the smooth muscle which lines arteries; the thyroid gland; cutaneous glandular tissue; the adrenal glands; the synovium and synovial fluid; and, the prostate and breast tissue; as well as the lining of the small and large intestines, the serotonin glands within both the brain and the gastrointestinal tract, substantia nigra, and synovial fluid-producing glands.

As a result of this common antigen, autoimmune targeting of the common antigen in the granules of otherwise normal secretory tissue results in inflammation. All human secretory tissue has a common secretory granule; therefore, all secretory tissue in the human body is a potential target. What may begin as a chronic, but localized, inflammatory reaction within the nasal mucosa can eventually evolve into systemic autoimmune inflammation with the autoimmune targeting of, and resulting inflammation within, different secretory tissues.

Without being bound by any theory or mechanism of action, contacting inhalant antigens with nasal mucosa initiates T-cell mediated, delayed hypersensitivity reactions that are a significant cause of chronic human inflammatory disease and other secondary diseases. This extends way beyond just the treatment of chronic sinusitis, in some embodiments. The manner by which disease occurs appears to involve the initiation of an autoimmune response directed toward secretory granules, which are present within the secretory tissue of the sinus and nasal mucous membrane and which are also present within all secretory tissue in the human body.

Without intending to be bound by any theory or mechanism of action, when individuals experience repeated delayed hypersensitivity reactions to inhaled foreign antigens, such as fungal survival spore antigens, over extended periods of time, such as years to decades, the initial reaction appears to occur at an inter-nasal, or inter-sinus, location adjacent to secretory tissue which is, therefore, adjacent to secretory granules. The proximity of the inflammatory reaction to secretory granules seems to occur because the density of secretory tissue in the sinus mucous membrane greatly exceeds that of any other secretory tissue in the human body. After many exposures and subsequent delayed hypersensitivity reactions to the inhaled foreign antigens, the subject's immune system appears to identify the signature of the inhaled antigen with the antigen-signature of the secretory granules, essentially identifying both as the foreign antigen, triggering the autoimmune response.

There are approximately 40 inhaled antigens used in standard SET testing. The inventors, however, have determined that only eight commonly elicit a delayed hypersensitivity reaction, including antigens from Alternaria sp., Aspergillus sp., Aspergillus niger, Candida sp., Chiadasporium sp., Curvularia specifera, Fusarium vasinfectum, Rhizopus sp., and Periplaneta sp. These antigens are also commonly found and, thus, are readily present in the environment as a source of antigens. Alternaria sp. is a genus of pervasive fungi which is implicated as a cause of rhinitis and bronchial asthma. Fungi in this genus are present on the surface of tomatoes, eggplant, potatoes, and houseplants, and may also be airborne. Aspergillus sp. is a very common airborne fungus which can cause clinical infections, particularly in immunosuppressed patients. This fungus is commonly found on marijuana leaves and is likely the fungus which produces the hallucinogen tetrahydrocannabinol by its role in the fermentation process. Candida sp., another pervasive fungus, is yeast which commonly causes vaginal infections and often colonizes patients' gastrointestinal tracts, and genitourinary tracts. Candida causes the most common and, therefore, the most fulminant, delayed hypersensitivity reactions. This fungus is used in most bakery products, in the process to produce Brie cheese, and in the fermentation process for some red wines. Aspergillus niger is known as “black mold” and is found on roofs, walls, bath tile grout, and shower heads of many buildings. This fungus is utilized in industrial fermentation and to ferment cider and wine and commonly causes delayed hypersensitivity reactions. Chiadosporium sp. is a genus of fungi that includes common indoor and outdoor molds. Curvularia spicifera is commonly found on leaves in the fall and in yard mulch. Fusarium vasinfectum is a micro-fungus found in soil, air, and on the surface of tomatoes, legumes, bananas and plant leaves, such as tobacco. This is likely one of the fungi involved in the fermentation process which produces nicotine when tobacco leaves undergo fungal fermentation. Rhizopus sp. is a genus of fungi found on the surface of bread, tobacco leaves, leather, and in jellies and syrup. Rhizopus is another fungus which likely produces nicotine by fermentation. Periplaneta sp. is the genus of cockroaches. Trichophyton sp. is the genus of fungi associated with athlete's foot, ringworm, and jock itch.

The compositions and formulations taught herein have been found useful in treating inflammations of tissue, particularly mucosal tissue. In some embodiments, they can be administered to a subject for treating inflammations of any tissue such as, for example, mucosal inflammations in the subject to prevent, treat, inhibit, or ameliorate the symptoms associated with the inflammation. For example, the compositions or formulations are also useful in treating autoimmune, or autoimmune-mediated, disorders. In some embodiments, they can be administered for treating these disorders of any tissue including, for example, chronic rhinosinusitis, Crohn's disease, rheumatoid arthritis, colitis, psoriasis, and ankylosing spondylitis, to name a few, with the goal of preventing, treating, inhibiting, and/or ameliorating the symptoms of any such condition. Other autoimmune, and autoimmune-related, diseases that can be treated using the methods taught herein include, but are not limited to Addison's disease, cardiomyopathy, high cholesterol, celiac disease, Graves' disease, lupus, Meniere's disease, multiple sclerosis, myositis, and scleroderma, and combinations thereof. One of skill will appreciate that most any autoimmune, or autoimmune related, disorder can be treated using the methods taught herein.

### Making and Administering the Agents and Formulations

Making the Formulations

In some embodiments, the compositions and formulations taught herein can be used in the making of a medicament that is used to prevent, treat, ameliorate the symptoms of, or even cure, an acute or chronic autoimmune, or autoimmune-mediated, condition. In some embodiments, the compositions and formulations can be used to reduce mucosal tissue inflammation, dysfunction, or damage. In some embodiments, the condition is chronic rhinosinusitis. The agents and formulations taught herein are suitable for use in a human subject meaning, for example, they fall within toxicity standards and regulations for their intended uses. The guidelines for use can follow the U.S. FDA regulations. In some embodiments, for example, the formulations taught herein have (i) an acute oral toxicity with an LD50 of at least 49,700 mg/kg or (ii) an acute dermal toxicity of at least 5000 mg/kg.

An agent, combination of agents, or a formulation taught herein can be designed for administration to a particular target site. For example, the target site can be the ostiomeatal complex of a subject. The design of the formulations can include, for example, (i) identifying the condition or use; (ii) identifying the target site; (iii) selecting components for the composition or formulation; and (iv) matching a dosage form for administration to the target site.

### Condition and Target

The condition can include any condition taught herein, for example, chronic sinusitis, or any other disorder taught herein, including any autoimmune or autoimmune-related condition. Identifying the target site includes, for example, select a target tissue for treatment, such as a healthy or damaged tissue at which the formulation can be applied to help prevent the onset of, inhibit, otherwise treat, or ameliorate the symptoms of, a condition. As taught herein, the ostiomeatal complex is an example of a desired target site.

### Components

### Antifungals

Although the combination of itraconazole and ketoconazole have been found to be surprising effective in the formulations taught herein, a person skilled in the art may also choose any other combination of antifungals suitable to kill, or at least inhibit or stop production of, the survival spores of concern herein. For example, the following antifungals may be of interest to the skilled artisan:

### Polyene Anti Fungals; Polyene Antimycotic

A polyene is a molecule with multiple conjugated double bonds. A polyene antifungal is a macrocyclic polyene with a heavily hydroxylated region on the ring opposite the conjugated system. This makes polyene antifungals amphiphilic. Examples include Amphotericin B; Candicidin; Filipin-35 carbons, binds to cholesterol (toxic); Hamycin; Natamycin-33 carbons, binds well to ergosterol; Nystatin; and Rimocidin.

### Imidazole, Triazole, and Thiazole Antifungals

Azole antifungal drugs (except for abafungin) inhibit the enzyme lanosterol 14 α-demethylase; the enzyme necessary to convert lanosterol to ergosterol. Depletion of ergosterol in fungal membrane disrupts the structure and many functions of fungal membrane leading to inhibition of fungal growth.
Imidazoles: Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole;
Triazoles: Albaconazole, Efinaconazole, Epoxiconazole, Fluconazole, Isavuconazole, Itraconazole, Posaconazole, Propiconazole, Ravuconazole, Terconazole, Voriconazole
Thiazoles: Abafungin
Allylamines: Allylamines inhibit squalene epoxidase, another enzyme required for ergosterol synthesis. Examples include Amorolfin, Butenafine, Naftifine, and Terbinafine.
Echinocandins: they inhibit the synthesis of glucan in the cell wall via the enzyme 1,3-Beta-glucan synthase. Examples include Anidulafungin, Caspofungin, Micafungin,
Others: Benzoic acid—has antifungal properties, but should be combined with a keratolytic agent; Ciclopirox—(ciclopirox olamine); Flucytosine or 5-fluorocytosine—an antimetabolite pyrimidine analog; Griseofulvin—binds to polymerized microtubules and inhibits fungal mitosis; Haloprogin—discontinued due to the emergence of more modern antifungals with fewer side effects; Tolnaftate—a thiocarbamate antifungal, which inhibits fungal squalene epoxidase (similar mechanism to allylamines like terbinafine); Undecylenic acid—an unsaturated fatty acid derived from natural castor oil; fungistatic, antibacterial, antiviral, and inhibits Candida morphogenesis; Crystal violet—a triarylmethane dye, it has antibacterial, antifungal, and anthelmintic properties and was formerly important as a topical antiseptic; Balsam of Peru has antifungal properties.
Any combination of the above corticosteroids can be used.

### Antibiotic

Tobramycin, and in cases where MRSA is present, vancomycin, has been found to be highly effective, particularly in combination with the compositions and formulations taught herein. These are aminoglycoside and glycoprotein types of antibiotics.

One of skill may prefer to use any known antibiotic of choice that is suitable for the conditions and administrations taught herein. Examples of aminoglycosides that can be used include gentamicin, tobramycin, amikacin, streptomycin, neomycin, paromomycin, and plazomicin. Examples of glycopeptides that can be used include vancomycin, teicoplanin, telavancin, ramoplanin, decaplanin, and bleomycin.

### Corticosteroids

Any topical steroid in Groups I-VII can be used, in some embodiments. Group I corticosteroids are up to 600 times stronger than hydrocortisone and include, but are not limited to, clobetasol propionate, betamethasone dipropionate, halobetasol propionate, and diflorasone diacetate. Group II corticosteroids include, but are not limited to, fluocinonide, halcinonide, amcinonide, and desoximetasone. Group III corticosteroids include, but are not limited to triamcinolone acetonide, mometasone furoate, fluticasone propionate, betamethasone dipropionate, and halometasone. Group IV corticosteroids include, but are not limited to, fluocinolone acetonide, hydrocortisone valerate, hydrocortisone butyrate, flurandrenolide, triamcinolone acetonide, and mometasone furoate. Group V corticosteroids include, but are not limited to, fluticasone propionate, desonide, fluocinolone acetonide, and hydrocortisone valerate. Group VI corticosteroids include, but are not limited to, alclometasone dipropionate, triamcinolone acetonide, fluocinolone acetonide, and desonide. Group VII corticosteroids include, but are not limited to, Any combination of the above corticosteroids can be used. hydrocortisone 2.5% and hydrocortisone 1%.

Once the use, the target site, the formulation, and the method of administration have been chosen, one of skill can readily select a dose, which will vary according to any of a variety of factors known the person of skill including, but not limited to, environmental conditions present at the site of use, for example, sunlight, heat, water, pH, gastric acids, and the like. In some embodiments, this formulation can be administered for uses in animals that are non-humans.

The compositions and formulations taught herein can be in a dosage form for administration topically for any use set-forth herein, including administration to the mucous membranes, particularly the ostiomeatal complex, and perhaps the gastrointestinal tract. Generally speaking, at least in some embodiments, an agent, combination of agents, or formulation taught herein, can be administered to a human secretory tissue. As discussed, the particular target tissue of interest, at least in some embodiments, is the ostiomeatal complex of a subject. For topical administration, in some embodiments, suitable formulations may include a biocompatible oil, wax, gel, powder, emulsion, polymer, or other liquid or solid carriers. Such formulations may be administered by applying directly to affected tissues.

The compositions and formulations taught herein can be administered in dosage units. The term “dosage unit” can refer to discrete, predetermined quantities of a compound that can be administered as unitary dosages to a subject. A predetermined quantity of active compound can be selected to produce a desired therapeutic effect and can be administered with a pharmaceutically acceptable carrier. The predetermined quantity in each unit dosage can depend on factors that include, but are not limited to, (a) the unique characteristics of the active compound and the particular therapeutic effect to be achieved, and (b) the limitations inherent in the art of creating and administering such dosage units. A “pharmaceutically acceptable carrier” is a diluent, adjuvant, excipient, or vehicle with which the composition is administered. A carrier is pharmaceutically acceptable after approval by a state or federal regulatory agency or listing in the U.S. Pharmacopeial Convention or other generally recognized sources for use in subjects. The pharmaceutical carriers include any and all physiologically compatible solvents, dispersion media, coatings, antibacterial and antifungal agents, isotonic and absorption delaying agents, and the like. Examples of pharmaceutical carriers include, but are not limited to, sterile liquids, such as water, oils and lipids such as, for example, phospholipids and glycolipids. These sterile liquids include, but are not limited to, those derived from petroleum, animal, vegetable or synthetic origin such as, for example, peanut oil, soybean oil, mineral oil, sesame oil, and the like.

Suitable pharmaceutical excipients include, but are not limited to, starch, sugars, inert polymers, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. In some embodiments, the composition can also contain minor amounts of wetting agents, emulsifying agents, pH buffering agents, or a combination thereof. Oral formulations, for example, can include standard carriers such as, for example, pharmaceutical grades mannitol, lactose, starch, magnesium stearate, sodium saccharine, cellulose, magnesium carbonate, and the like. See Martin, E. W. Remington's Pharmaceutical Sciences.

In some embodiments, the compositions or formulations can be designed for administration as a sustained release formulation, and the formulation can include one or more agents in addition to the compositions taught herein. In some embodiments, the sustained release formulations can reduce the dosage and/or frequency of the administrations of such agents to a subject.

In some embodiments, the compositions or formulations can be filtered to remove particles. For example, a composition can be re-dissolved, and brought to a suitable concentration for filtration to further remove unwanted materials. In some embodiments, the suitable concentration for filtration can be about 1%, about 2%, about 3%, about 4%, about 5%, about 6%, about 7%, about 8%, about 9%, about 10%, about 12%, about 14% or any concentration therein in increments of 0.2%.

In some embodiments, the compositions or formulations can be administered as a liquid in solution or mixture. In some embodiments, the compositions or formulations are prepared as pharmaceutically acceptable emulsions. In some embodiments, the emulsifying includes adding a pharmaceutically acceptable oil to a liquid formulation to create the emulsion.

Surfactants can be used in the formulations taught herein. Example surfactants include anionic, cationic, zwitterionic, and nonionic surfactants. An example of cationic surfactants includes sodium dodecylsulfate (SDS). An example of anionic surfactants includes cetylpyridinium bromide. An example of zwitterionic surfactants includes dipalmitoylphosphatidylcholine (lecithin). An example of noninionic surfactants includes polyoxyethylene(4) lauryl ether (BRIJ 30). In some embodiments, SPAN- or TWEEN-type surfactants can be used, including SPAN 60, SPAN 80, TWEEN 60, and TWEEN 80. In fact, any one or any combination of the numerous surfactants known to one of skill can be used to the extent that there is reason to believe that such use would be consistent with the teachings provided herein.

In some embodiments, the pH of the formulations can be adjusted. In some embodiments, the pH can be 3.8, 4.0, 4.2, 4.4, 4.6, 4.8, 5.0, 5.2, 5.4, 5.6, 5.8, 6.0, 6.2, 6.4, 6.6, or any increment of about 0.1 therein.

In some embodiments, the viscosity of the formulations can be adjusted. The viscosity of the formulations is an improvement over the art, as the viscosity can be adjusted. For example, the viscosity can be adjusted by choice of solvent or oil, for example, or temperature used in processing as, generally, temperature effect on viscosity is temporary. In some embodiments, the viscosity can be adjusted by warming a desired oil, as long as the heat is not so high as to cause degradation of the formulation. In some embodiments, an oil can be heated up to about 65° C., about 55° C., about 45° C., about 35° C., or any temperature therein in increments of about 1° C.

In some embodiments, the compositions or formulations can include particles for administration. In such embodiments, the particles can have mass median aerodynamic diameters ranging from about 1 micron to about 50 microns, from about 5 microns to about 40 microns, from about 5 microns to about 30 microns, from about 7 microns to about 25 microns, from about 10 microns to about 20 microns, or any range therein. In some embodiments, the carrier can include agglomerates of the particles. In some embodiments, at least 25 percent, 30 percent, 40 percent, 50 percent, 60 percent, 70 percent, 80 percent, 85 percent, 90 percent, 95 percent, 97 percent, 98 percent, 99 percent, 99.5 percent, 99.7 percent, 99.9 percent, or any range therein, of such particles composes the total mass of the pharmaceutical composition.

In some embodiments, the compositions or formulations can be delivered as a dry powder containing particles as indicated above, the particles may be individual particles or agglomerates of particles. The agglomerates can be the result of a controlled agglomeration process or they may simply be the result of the intimate contact of the powder particles. In either case, it is desired that the nasally-administered particles are capable of being delivered to the ostiomeatal complex.

As stated above, excipients may be included in the pharmaceutically acceptable compositions. Excipients may be included, for example, in order to dilute the powder to an amount which is suitable for delivery from the particular intended powder inhaler; to facilitate the processing of the preparation; to improve the powder properties of the preparation; to improve the stability of the preparation, for example, by adding antioxidants or pH-adjusting compounds; or to add a taste to the preparation. Any excipient should not adversely affect the stability of, or disadvantageously interfere with an active agent. The pharmaceutically acceptable composition should also be stable, have a controlled amount of hygroscopicity, and have good powder properties without causing adverse effects on the subject's tissue.

Examples of excipients can include mono-, di-, and polysaccharides, sugar alcohols and other polyols, such as for example lactose, glucose, raffinose, melezitose, lactitol, maltitol, trehalose, sucrose, mannitol and starch. Depending upon the inhaler or other device to be used, the total amount of such excipients may vary over a very wide range. In some embodiments, little or no excipient would be required, whereas in embodiments that require large powder volumes, a very high percentage of excipient may be necessary. One of skill can determine the amount and type of excipient needed through routine experimentation.

A dry powder preparation can be manufactured in several ways, using conventional techniques. For example, it may be necessary to grind the active agent and, if appropriate, combine the active agent with the carrier in a suitable grinding mill such as, for example, in a jet mill. The active agent and carrier can be dry mixed and then milled together. Or, alternatively the active agent and carrier can be ground separately and then mixed. Separate grinding and mixing may be necessary, for example, where the active agent and carrier have different physical properties, such as hardness and brittleness, and resistance to grinding.

In some embodiments, better mixing may be accomplished by first dissolving the active agent and carrier in a suitable solvent such as, for example, water, to form a solution. This procedure allows the artisan to adjust the pH-value to a desired level. For example, a pH range of about 5.0 to about 8.5 is generally accepted as the limit for inhalation products, and this should be taken into account in the preparation of the pharmaceutical compositions. The solvent is removed from the solution to form a powder, and suitable drying methods can include vacuum concentration, open drying, spray drying, freeze drying and use of supercritical fluids. Temperature limits should be considered, for example, where active agent materials can degrade and lose activity above a known temperature limit. After drying, the solid precipitate can, if necessary, be ground to obtain the desired particle size and particle size distribution.

The powders can be processed to improve flow properties, for example, by dry granulation to have superior handling characteristics that complement an inhaler device. In fact, a particular inhaler can be configured to ensure that the particles or agglomerates entering the respiratory tract of the patient are largely within the desired size range. In addition to spray drying and jet milling, other methods such as spray freeze-drying into liquids, supercritical fluid technology, and crystal engineering can be used to produce desired particles, in some embodiments.

In some embodiments, the pharmaceutical composition includes a material that is in the form of a dry powder and suitable for inhalation in which at least 50% of the total mass consists of particles having mass median aerodynamic diameters ranging from about 1 microns to about 50 microns, from about 5 microns to about 50 microns, from about 5 microns to about 40 microns, from about 5 microns to about 30 microns, from about 7 microns to about 25 microns, from about 10 microns to about 20 microns, or any range therein. In some embodiments, the pharmaceutical composition can include agglomerates of the particles. In some embodiments, at least 25 percent, 30 percent, 40 percent, 50 percent, 60 percent, 70 percent, 80 percent, 85 percent, 90 percent, 95 percent, 97 percent, 98 percent, 99 percent, 99.5 percent, 99.7 percent, 99.9 percent, or any range therein, of such particles composes the total mass of the pharmaceutical composition.

One of skill will appreciate that the particle size of a delivered powder is an important control parameter of the delivered of the active agent or formulation. Particle size is often expressed in terms of mass median aerodynamic diameter (MMAD), a parameter that is based on particle size, shape, and density. For a spherical particle, the MMAD is equal to mass median diameter (MMD) times the square root of density. For a non-spherical particle, MMAD is equal to MMD times the root of (p/x), in which X is the shape factor. Thus, particles with a smaller than unit density will have an MMAD that is smaller than their MMD. Ordinarily, the efficient intranasal delivery requires that the particles are small enough to pass to the target tissue which, in this case is the ostiomeatal complex. In the methods taught herein, however, the particle sizes are selected to cause the particles to deposit in the ostiomeatal complex. The nose acts as a series of filters for inhaled particles, and particles having an MMAD of greater than 100 microns can be trapped generally. Particles having an MMAD of greater than 10 microns can come into contact with mucous membranes of the sinuses. Particles having an MMAD ranging from 5 to 10 microns are deposited in the large ciliated airways, and particles having an MMAD ranging from 1 to 5 microns are typically expected reach the alveoli of the lungs. It should be appreciated by one of skill that a variety of methods can be used to measure particle size. Such methods include, but are not limited to, the Anderson Cascade Impactor (ACI), the New Generation Impactor (NGI), and time-of-flight (TOF) analyses.

Administering

The agents and formulations can be applied to a target tissue non-parenterally, including oral, sublingual, topical, transdermal, ophthalmic, otic, nasal, rectal, and vaginal routes. In some embodiments, the compositions or formulations can be applied at least topically using, for example, irrigation or nebulization. In many embodiments, the administration includes intranasal delivery of the topical administration of the agents and formulations taught herein.

In some embodiments, the intranasal delivery can include any method of delivering an agent or formulation taught herein to the ostiomeatal complex. Examples of intranasal delivery include snorting, use of syringe or dropper, delivery through a squeeze bottle, use of a sprayer or atomizer such as an inhaler or a nebulizer.

Any administration vehicle known to one of skill to be suitable for administration of the compounds, compositions, and formulations taught herein can be used. A “vehicle” can refer to, for example, a diluent, excipient or carrier with which a compound is administered to a subject.

The terms “administration” or “administering” can be used to refer to a method of incorporating a composition into or onto the cells or tissues of a subject, either in vivo or ex vivo to test the activity of a system, as well as to diagnose, prevent, treat, or ameliorate a symptom of a disease or condition. In one example, a compound can be administered to a subject in vivo using any means of administration taught herein. In another example, a compound can be administered ex vivo by combining the compound with cell tissue from the subject for purposes that include, but are not limited to, assays for determining utility and efficacy of a composition. And, of course, the compositions can be used in vitro to test their stability, activity, toxicity, efficacy, and the like. When the compound is incorporated in the subject in combination with one or more active agents, the terms “administration” or “administering” can include sequential or concurrent incorporation of the compound with the other agents such as, for example, any agent described above. A composition can be formulated, in some embodiments, to be compatible merely with its intended route of administration.

In some embodiments, the compositions or formulations can be administered in conjunction with at least one other therapeutic agent for the condition being treated. The amounts of the agents can be reduced, even substantially, such that the amount of the agent or agents desired is reduced to the extent that a significant response is observed from the subject. A “significant response” can include, but is not limited to, a reduction or elimination of a symptom, a visible increase in a desirable therapeutic effect, a faster response to the treatment, a more selective response to the treatment, or a combination thereof. In some embodiments, any therapeutic agent can be administered, for example, in an amount ranging from about 0.1 μg/kg to about 1 mg/kg, from about 0.5 μg/kg to about 500 μg/kg, from about 1 μg/kg to about 250 μg/kg, from about 1 μg/kg to about 100 μg/kg from about 1 μg/kg to about 50 μg/kg, or any range therein.

Generally speaking, an agent administered herein might include an anti-inflammatory agent such as, for example, aspirin or an NSAID; an antimicrobial agent such as, for example, an antibacterial, an antifungal, an antiviral, or an antiseptic; an analgesic; an anesthetic; a steroid; an antihistamine a terpene; a fragrant; a flavoring agent; an inorganic salt; a sugar; or a combination thereof.

In some embodiments, any suitable anti-inflammatory agent can be used, and many of the most useful anti-inflammatory agents also have analgesic and/or antipyretic properties. Examples include, for example, acetaminophen, aspirin (acetylsalicylic acid), ibuprofen, phenylbutazone, indomethacin, sulindac, diclofenac, and naproxen. In contrast to NSAIDs, steroids can be used in some embodiments.

In some embodiments, any microbial agents can be used. Examples include, but are not limited to antibacterials, antifungals, antivirals, and other topical antiseptics. Examples of antibacterial agents (antibiotics) include the penicillins such as, for example, penicillin G, ampicillin, methicillin, oxacillin, and amoxicillin; the cephalosporins such as, for example, cefadroxil, ceforanid, cefotaxime, and ceftriaxone; the tetracyclines such as, for example, doxycycline, minocycline, and tetracycline; the aminoglycosides such as, for example, amikacin, gentamycin, kanamycin, neomycin, streptomycin, and tobramycin; the macrolides such as, for example, azithromycin, clarithromycin, and erythromycin; the fluoroquinolones such as, for example, ciprofloxacin, lomefloxacin, and norfloxacin; and other antibiotics including chloramphenicol, clindamycin, cycloserine, isoniazid, rifampin, and vancomycin.

In some embodiments, any antiviral agents can be used. Examples includel-D-ribofuranosyl-1, 2,4-triazole-3 carboxamide; 2-hydroxy-ethoxy methylguanine; adamantanamine; 5-iodo-2′-deoxyuridine; trifluorothymidine; interferon; adenine arabinoside; protease inhibitors; thymadine kinase inhibitors; sugar or glycoprotein synthesis inhibitors; structural protein synthesis inhibitors; attachment and adsorption inhibitors; and nucleoside analogues such as acyclovir, penciclovir, valacyclovir, and ganciclovir.

In some embodiments, any antifungal agents can be used. Examples include both fungicidal and fungistatic agents such as, for example, amphotericin B, butylparaben, clindamycin, econaxole, fluconazole, flucytosine, griseofulvin, nystatin, and ketoconazole. In some embodiments, fluconazole is not used.

In some embodiments, any topical antiseptic, analgesic, or anesthetic can be used. Examples of topical antiseptics include povidone-iodine and benzalkonium chloride. Examples of analgesics or anesthetics include, for example, a terpene or sesquiterpene, such as menthol; benzocaine; bupivacaine; butambenpicrate; chlorprocaine; cocaine; dibucaine; dimethisoquin; dyclonine; etidocaine; hexylcaine; hexylresorcinol; ketarine; lidocaine; mepivacaine; phenol; phenolate; piperocaine; pramoxine; procaine; ropavacaine; tetracaine; tripelennamine; xylocaine; and pharmaceutically acceptable salts thereof, such as dimethisoquin hydrochloride and pramoxine hydrochloride). Other examples of analgesics include opioids such as, for example, morphine, codeine, hydrocodone, and oxycodone. Examples of other therapeutic agents can include, but are not limited to, a local anesthetic agent, for example, benzocaine, cocaine, lidocaine, prilocaine, in amounts of perhaps 1-10%, 2-8%, 3-6%, 2-4%, or any range therein, to reduce discomfort in the sinuses.

In some embodiments, an anti-infective agent can be used and may include amylmetacresol, benzalkonium, cetylpyridinium, chlorhexidine, dequilinium, domiphen, dichlorobenzyl alcohol, phenol, or tyrothicin. In some embodiments, an agent can be selected to treat snoring and some forms of sleep apnea and can be, for example, a moisturizer or humectant including, but not limited to, hyaluronic acid, glycerol, sorbitol, or a poly(ethylene glycol), to decongest and lubricate the pharynx and allow for the passage of air and improved respiration of the subject. Examples of such active agents can be found in US Published Application No. 20070218114.

An oral administration of agents can be combined with the topical administration of the agents and formulations, as taught herein. In some embodiments, the methods further comprise orally administering an effective amount of an agent taught herein to a subject in combination with the topical administration to a target tissue, such as the ostiomeatal complex. In some embodiments, the teachings are directed to a method of treating an inflammation of a tissue of subject, the method comprising administering an effective amount of a composition taught herein to a tissue of the subject.

An “effective amount” of a compound can be used to describe a therapeutically effective amount or a prophylactically effective amount. An effective amount can also be an amount that ameliorates the symptoms of a disease. A “therapeutically effective amount” can refer to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect. In some embodiments, the therapeutically effective amount should be administered in an amount sufficient to result in amelioration of one or more symptoms of a disorder; prevention of the advancement of a disorder; regression followed by recidivism of a disorder and, thus, recurrence of a disorder. In some embodiments, for example, a therapeutically effective amount can refer to the amount of an agent that provides a measurable response of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of a desired action of the composition. For example, a therapeutically effective amount can, in some embodiments, can remove recurrence of a condition or disorder entirely, such that the measurable response to the treatment was 100%. In some embodiments, treatments are repeatedly administered over time, such that the measurable response is less than 100%, in any amount taught above, but repeated administrations of an agent or combination of agents taught herein at least inhibit, and possibly remove, any recurrence of the condition or disorder in the subject.

In cases of the prevention or inhibition of the onset of a disease or disorder, or where an administration is considered prophylactic, a prophylactically effective amount of a composition or formulation taught herein can be used. A “prophylactically effective amount” can refer to an amount that is effective at the dosages and periods of time necessary to achieve a desired prophylactic result. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

One of skill understands that the amount of the agents administered can vary according to factors such as, for example, the type of disease, age, sex, and weight of the subject, as well as the method of administration. Dosage regimens may also be adjusted to optimize a therapeutic response. In some embodiments, a single bolus may be administered; several divided doses may be administered over time; the dose may be proportionally reduced or increased; or, any combination thereof, as indicated by the exigencies of the therapeutic situation and factors known to one of skill in the art. It is to be noted that dosage values may vary with the severity of the condition to be alleviated, as well as whether the administration is prophylactic, such that the condition has not actually onset or produced symptoms. Dosage regimens may be adjusted over time according to the individual need and the professional judgment of the person administering or supervising the administration of the compositions, and any dosage ranges set forth.

In some embodiments, fluconazole may be administered in amounts of 100 mg (oral) twice daily for 10 days, then daily for 7 days; terbenafine (an aminoglycoside antibiotic) may be administered in amounts of 250 mg (oral) daily for 21 days; and nebulized itraconazole (0.2%)+ketaconazole (0.2%) may be administered for sinus irrigation.

An administration schedule should be designed to kill the targeted fungal survival spores. In some embodiments, for example, an effective amount of an antibiotic is combined with an effective amount of a first antifungal, an effective amount of a second antifungal, and an effective amount of a corticosteroid. The formulation can be topically administered intranasally to the ostiomeatal complex of a subject twice daily for an first administration period of at least about one week, two weeks, three weeks, or four weeks. In some embodiments, the first administration period can be stopped for a rest period of about 1 day, 2 days, 3 days, 4 days, 5 days, 6 days, 7 days, 8 days, 9 days, 10 days, 11 days, 12 days, 13 days, or 14 days, and then resumed at once per day for at least an additional about one week, two weeks, three weeks, or four weeks.

As a topical administration, any dose taught herein can be carried to a subject's ostiomeatal complex using delivery device and any suitable volume of carrier chosen by one of skill to carry the agents to the target tissue. In some embodiments, the delivery device can function to irrigate the target tissue area or nebulize the target tissue area. The formulations taught herein can be carried with an aqueous carrier or non-aqueous carrier and can be intended for use with one or more administrations. For example a water carrier can have a volume ranging from about 1 ml to about 100 ml, from about 1 ml to about 50 ml, from about 1 ml to about 40 ml, from about 1 ml to about 30 ml, from about 1 ml to about 20 ml, from about 1 ml to about 10 ml, from about 2 ml to about 5 ml, or any range or amount therein in increments of 0.1 ml. In some embodiments, the carrier can be intended for use in a single administration and can have a volume of about 0.5 ml, about 1.0 ml, about 2.0 ml, about 3.0 ml, about 4.0 ml, about 5.0 ml, about 6.0 ml, about 7.0 ml, about 8.0 ml, about 9.0 ml, about 10.0 ml, or any volume therein in increments of 0.1 ml. In some embodiments, for example, a formulation can include about 125 mg tobramycin, about 40 mg ketoconazole, about 40 mg itraconazole, about 0.6 mg mometasone, and these components are carried in a total aqueous volume of about 1-5 ml, for example about 3 ml, for a single administration. In some embodiments, for example, a formulation can include about 160 mg vancomycin, about 40 mg ketoconazole, about 40 mg itraconazole, about 0.6 mg mometasone, and these components can be carried in a total aqueous volume of about 1-5 ml, for example about 3 ml, for a single administration. It should be appreciated that any formulation taught herein can be carried in any of the volumes taught herein.

In some embodiments, for example, a formulation can be made comprising from about 40 mg to about 375 mg, and preferably about 125 mg tobramycin or 160 mg vancomycin; from about 13 mg to about 120 mg, and preferably about 40 mg, of itraconazole; from about 13 mg to about 120 mg, and preferably about 40 mg, of ketoconazole; and, from about 0.2 mg to about 5.4 mg, and preferably from about 0.6 mg to about 1.8 mg, and any range or amount therein in increments of 0.1 mg, of mometasone furoate. These components can be carried in a total aqueous volume of about 1-5 ml, for example about 3 ml, for a single administration. The formulation can be topically to any affected tissue. For example, the formulation can be administered intranasally to the ostiomeatal complex of a subject twice daily for an first administration period of at least about one week, two weeks, three weeks, or four weeks. In some embodiments, the first administration period can be stopped for a rest period of about 1 day, 2 days, 3 days, 4 days, 5 days, 6 days, 7 days, 8 days, 9 days, 10 days, 11 days, 12 days, 13 days, or 14 days, and then resumed at once per day for at least an additional about one week, two weeks, three weeks, or four weeks.

In fact, in some embodiments, since the agents in the formulation are either not absorbed, or are absorbed in clinically insignificant, any number of administrations are theoretically possible although impractical. The maximum number of administrations for patient compliance might be 1, 2, 3, 4, or 5 times per day. In some embodiments, the minimum number of administrations of a formulation taught herein can be once a day, every 3 days for a desired period time which can be, for example, 10 days, 12 days, 14 days, 16 days, 18 days, 20 days, 22 days, 24 days, 26 days, 28 days, 30 days, 32 days, 34 days, 36 days, 38 days, 40 days, 42 days, 44 days, 46 days, 48 days, 50 days, 52 days, 54 days, 56 days, 58 days, 60 days, or any number of days therein in increments of 1 day. In some embodiments, the formulations can be administered for a total period of 1 month, 2 months, 3 months, 4 months, 5 months, 6 months, 7 months, 8 months, 9 months, 10 months, 11 months, 12 months, or any duration therein in increments of 1 day.

In some embodiments, the administration can be done to treat an episode that may be triggered by an isolated event, such as exposure to food or drink, for example, that may carry an antigen that can be inhaled. For example, red wine and brie are known to be carriers of Candida species of fungi, that can result in fungal spores in a subjects ostiomeatal complex. In some embodiments, the administration can be done to treat for a seasonal exposure due to environmental conditions that are conduce to the transmission of fungi, such as humid conditions. Sometimes, the environmental conditions are planned, such as with airline travel during which fungal spores can be recirculated and recirculated in the air during the flight through the ventilation systems, making inhalation of the antigens likely. Sometimes the exposure is intended by the subject, and the formulations taught herein can be administered prophylactically. In some embodiments, a prophylactic administration can include delivering the compositions or formulations taught herein to the ostiomeatal complex both before and after exposure. The administration can occur, for example from 1 to 5 times before the exposure and/or 1 to 5 times following the exposure for such treatment. Sometimes, the exposure is unintended, and prophylactic measures can still be taken using the methods taught herein.

Likewise, combination therapies with these compositions and formulations, and certainly any other agents taught herein, can be administered, for example, for 30 minutes, 1 hour, 2 hours, 4 hours, 8 hours, 12 hours, 18 hours, 1 day, 2 days, 3 days, 4 days, 5 days, 6 days, 7 days, 8 days, 9 days, 10 days, 2 weeks, 3 weeks, 4 weeks, 6 weeks, 3 months, 6 months 1 year, any combination thereof, or any amount of time considered desirable by one of skill. The agents can be administered concomitantly, sequentially, or cyclically to a subject. Cycling therapy involves the administering a first agent for a predetermined period of time, administering a second agent or therapy for a second predetermined period of time, and repeating this cycling for any desired purpose such as, for example, to enhance the efficacy of the treatment. The agents can also be administered concurrently. The term “concurrently” is not limited to the administration of agents at exactly the same time, but rather means that the agents can be administered in a sequence and time interval such that the agents can work together to provide additional benefit. Each agent can be administered separately or together in any appropriate form using any appropriate means of administering the agent or agents. One of skill can readily select the frequency, duration, and perhaps cycling of each concurrent administration.

In some embodiments, the combination therapy can include any combination of agents with the formulations taught here, for example, the systemic administration of an antibiotic, antifungal, or anti-inflammatory. In some embodiments, the combination therapy can include administration of an antiproliferative, an antineoplastic, an antimitotic, an anti-inflammatory, an antiplatelet, an anticoagulant, an antifibrin, an antithrombin, an antibiotic, an antiallergic, an antioxidant, and any prodrugs, codrugs, metabolites, analogs, homologues, congeners, derivatives, salts and combinations thereof. In some embodiments, the combination therapy can include administration of an immunomodulatory agent.

In some embodiments, a therapeutically or prophylactically effective amount of any active agent taught herein, whether it be a topical administration, or a systemic administration, of an active agent, taught herein, may range in concentration from about 0.01 nM to about 10 M; from about 0.01 nM to about 5 M; from about 0.01 nM to about 1.0 M; from about 0.01 nM to about 0.5 M; from about 0.01 nM to about 0.10 M; from about 0.01 nM to about 0.05 M; from about 0.1 nM to about 150 nM; from about 0.1 nM to about 500 μM; from about 0.1 nM to about 1000 nM, 0.001 μM to about 0.10 M; from about 0.001 μM to about 0.5 M; from about 0.01 μM to about 150 μM; from about 0.01 μM to about 500 μM; from about 0.01 μM to about 1000 nM, or any range or amount therein in increments of 0.01 nM. In some embodiments, the compositions may be administered in an amount ranging from about 0.005 mg/kg to about 100 mg/kg; from about 0.005 mg/kg to about 400 mg/kg; from about 0.01 mg/kg to about 300 mg/kg; from about 0.01 mg/kg to about 250 mg/kg; from about 0.1 mg/kg to about 200 mg/kg; from about 0.2 mg/kg to about 150 mg/kg; from about 0.4 mg/kg to about 120 mg/kg; from about 0.15 mg/kg to about 100 mg/kg, from about 0.15 mg/kg to about 50 mg/kg, from about 0.5 mg/kg to about 10 mg/kg, or any range or amount therein in increments of 0.1 mg/kg, wherein a human subject is often assumed to average about 70 kg.

Articles of Manufacture

Kits that encompass finished, packaged and labelled products are provided. These are articles of manufacture that include the appropriate unit dosage form in an appropriate vessel or container that is hermetically sealed. Such vessels or containers can include an intranasal delivery device which might be selected, for example, from the group consisting of a syringe or dropper, a squeeze bottle, or perhaps a sprayer or atomizer such as an inhaler or a nebulizer.

As with any such product, the packaging material and container are designed to protect the stability of the product during storage and shipment. Storage of the agents and/or formulations can be in dry form or liquid form. In either form, concentration can be adjusted to vary potency. And, whether dry or liquid form, the agents and/or formulations can be stored in amber bottles for stability.

One of skill will appreciate that the compositions or formulations should remain stable, or at least substantially stable, until useful or activated, and this can relate to a shelf life, or a time between creation and administration of the composition, or some combination thereof. In some embodiments, the composition is stable, or substantially stable, when usable as intended within a reasonable amount of time, a time that is considered reasonable by one of skill for the applications taught herein. In some embodiments, the composition should be usable within a reasonable time from the making to the administration of the composition and, in some embodiments, the composition should have a reasonable commercial shelf life, a shelf life that is considered reasonable to one of skill. A reasonable shelf life can be at least 6 months, at least 1 year, at least 18 months, at least 2 years, at least 3 years, or any time in-between in increments of about 1 month, in some embodiments. In some embodiments, a composition or formulation can be considered as “stable” if it loses less than 10% of its original activity. In some embodiments, a composition or formulation can be considered as stable if it loses less than 5%, 3%, 2%, or 1% of its original activity. In some embodiments, a composition or formulation can be considered as “substantially stable” if it loses greater than about 10% of its original activity, as long as the composition can perform it's intended use to a reasonable degree of efficacy. In some embodiments, the composition can be considered as substantially stable if it loses activity at an amount greater than about 12%, about 15%, about 25%, about 35%, about 45%, about 50%, about 60%, or even about 70%. The activity loss can be measured by comparing activity at the time of packaging to the activity at the time of administration, and this can include a reasonable shelf life. In some embodiments, the composition is stable or substantially stable, if it remains useful for a period ranging from 3 months to 3 years, 6 months to 2 years, 1 year, or any time period therein in increments of about 1 month.

Moreover, the articles of manufacture can include instructions for use or other information material that can advise the user such as, for example, a physician, technician or patient, regarding how to properly administer the composition as a prophylactic, therapeutic, or ameliorative treatment of the disease of concern. In some embodiments, instructions can indicate or suggest a dosing regimen that includes, but is not limited to, actual doses and monitoring procedures.

In some embodiments, the instructions can include informational material indicating how to administer a composition for a particular use or range of uses, such as a particular indication taught herein, for example, as well as how to monitor the subject for positive and/or negative responses to the administration.

In some embodiments, the kits can be designed for physicians, patients, or over the counter use by any subject. In some embodiments, the kit is for treating any of the other indications taught herein, such that the kit can have instructions for use. The instructions can be designed for the physician, the patient, or any subject, including, for example, instructions for mixing the components for administration, suggested dilution factors for various target sites, and potential combination therapies for combined administrations, such as topical combined with oral administration. The suggested dilution factors can be selected from the administration ranges taught herein, for example, which can be modified in some embodiments as desired, and incorporated into the compositions.

Without intending to be limited to any theory or mechanism of action, the following examples are provided to further illustrate the teachings presented herein. It should be appreciated that there are several variations contemplated within the skill in the art, and that the examples are not intended to be construed as providing limitations to the claims.

### Example 1. Identification of the Fungal Antigens and Related Testing

Although the methods taught herein do not require the step of identifying the presence of any particular antigens, an identification of particular antigens helps to validate a reason for use of the methods provided herein, as well as to design the composition or formulation accordingly. The skin test is a valuable and unique skin test given to patients, as it can be used to observe the subject over a period of up to ten days, for example, to determine a “delayed” hypersensitivity to certain antigens of interest such as, for example, the Candida species and the Aspergillus species described herein. One of skill will appreciate that the antigen injected can be any antigen of interest, such as the species taught herein. The term “antigen” can be used to refer to any agent that stimulates an immune response in the subject being tested. For example, in some embodiments, such an agent can be a peptide, polypeptide or protein, which stimulates an immune response in a subject. An antigen may be inhaled by a subject, and can include, but is not limited to, Alternaria sp., Aspergillus sp., Aspergillus niger, Candida sp., Chladosporium sp., Curvularia specifera, Fusarium vasinfectum, Rhizopus sp., Penicillium sp., Periplaneta sp., Trichophyton sp. and similar sources of inhalable antigens known in the art.

The test is a skin reaction test that includes injecting an antigen into the skin of the subject at a test site and measuring observable symptoms. Test site injections are a “standard injection technique” used in serial endpoint titration (SET) testing. See, for example, Nadarajah, Ravi et al. Introduction to Serial Endpoint Titration. Immunology and Allergy Clinics 21(2):369-381(2001). Standard, FDA approved, injectable antigens are used. The uniqueness of this test, however, is that it is used in the methods taught herein and delivers an indication of “delayed” hypersensitivity by requiring an observation of the test site at least 24 hours after injection of the antigen to diagnose an antigen-mediated inflammation on the subject by viewing a positive reaction is observed at the test site. To observe the delayed hypersensitivity, serial dilutions of the antigen can be injected into the skin of the subject, and a positive reaction comprises a delayed hypersensitivity reaction to the antigen at least 24 hours post-injection, the reaction being an observable response at the site of injection measuring at least 6 mm in diameter. In some embodiments, the response is observed for an extended period after injection including, but not limited to, 24 hours, 36 hours, 48 hours, 60 hours, and 72 hours post-injection, and any post-injection time therein in increments of 0.1 hours. The term “antigen-mediated inflammation” can be used to refer to a set of sustained tissue-specific, organ-specific or systemic clinical symptoms associated with altered immune homeostasis. This can be manifested, for example, by qualitative and/or quantitative defects of expressed autoimmune responses. Antigen-mediated inflammation can include, but is not limited to, rheumatoid arthritis, cardiovascular disease and gastrointestinal inflammation, for example.

Injecting the Antigen

The test site for the injection and mapping can be selected by the skill artisan. For example, the test site can be the skin of one or both upper arms, the skin of one or both legs, the skin of the torso of the subject, or some combination of these sites. A daily assessment of the test site can be observed and, in some embodiments, the daily assessment can be made for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days.

In order to inject the antigen and determine a sensitivity level, the antigen needs to be placed into an injectable solution at various concentrations. In some embodiments, a base solution of about 0.5 mL of 1:10 antigen is placed in an appropriate solution that is easily selected by the skilled artisan. A diluent is needed for the dilutions. For example, one type of an acceptable diluent can be prepared to contain about 9.5 mL of 0.9% NaCl with trace amounts of albumin and phenol for use in adjusting the antigen concentration. A serial dilution can then be prepared for injection of the antigen at various concentrations. For example, the base solution may be diluted 5, 25, 125, 625, or 3125 times with the diluent.

The subject is then skin-tested by injecting the antigen at the one or more select test sites on the subject's skin. A subject's reaction to an antigen can occur in phases. An initial “acute phase histamine reaction” can occur within less than 10 minutes post-injection, can resolve in about 8 hours, and should resolve within 24 hours. Persistent or new reactions, such as skin eruptions that occur or last for more than 24 hours post-injection are considered “delayed hypersensitivity reactions.” The daily assessment could include daily photographs or video of the test site for analysis and review. In some embodiments, the assessment can include grading a delayed hypersensitivity reaction, such that the more robust the reaction, the more likely the antigen is capable of inducing inflammation in secretory tissue of the subject. For example, in one grading scale, mild reactions can measure from about 1 mm to about 5 mm in diameter, moderate reactions measure from about 6 mm to about 10 mm in diameter, and severe reactions measure from about 11 mm to 15 mm or greater in diameter. In some embodiments, a clearly labeled grid can be configured, and used, for mapping and grading of delayed hypersensitivity reactions. In some embodiments, an antigen can be identified as the cause of secretory tissue inflammation where the delayed hypersensitivity reaction measures at least 6 mm in diameter at least 24 hours post-injection.

The subject can maintain a detailed diary to record symptoms even if seemingly unrelated. Symptoms of interest can include, but are not limited to, severe fatigue/malaise, facial pain (symptoms of sinusitis); copious post-nasal drainage, secondary hoarseness, or pharyngitis; arthritis/fibromyalgia-type pain; gastrointestinal symptoms such as irritable bowel syndrome, reflux, and/or constipation; “Brain fog” or worsening of Alzheimer's disease symptoms; depression; nasal obstruction; worsening of snoring/sleep disorders; dizziness, vertigo, and tinnitus; worsening of psoriasis or rosacea; asthma or COPD symptoms; and, worsening of multiple sclerosis (MS), Parkinson's disease and amyotrophic lateral sclerosis (ALS) symptoms.

In some embodiments, the methods can include counseling the subject to eliminate certain foods or to avoid certain geographic areas with high concentrations of the antigen identified as causing the delayed hypersensitivity reaction.

In some embodiments, the methods can include counseling the subject to stay away from, prior to the antigen testing, non-steroidal anti-inflammatory drugs (NSAIDs), aspirin, acetaminophen, prescription and over the counter seasonal allergy medicine, steroids, and food and supplements that are high in salicylates, including, but not limited to, apples, berries, and cumin.

In some embodiments, it is desirable to obtain at least one additional diagnostic test both prior to and after injection of the antigen. The diagnostic test can include, for example, a test selected from the group consisting of prostate-specific antigen (PSA); C-reactive protein (CRP} levels; cholesterol (e.g., LDL, HDL and VLDL) levels; thyroid function; liver function; an immune response; an auto-immune marker; serum cortisol levels; and, a complete blood count. The term “immune response” can be used to refer to a change in the phenotype of a subject's immune system including, but not limited to, an increase in lymphocytes that recognize a particular antigen. The term “auto-immune marker” can be used to refer to rheumatoid factor, complement factor, antinuclear antibodies (ANA), inflammatory cytokines, down-regulation of regulatory T cells, and other markers known in the art. The term “thyroid function” includes, but is not limited to, levels of triiodothyronine (T3), thyroxine (T4), thyroid-stimulating hormone (TSH). The term “liver function” includes, but is not limited to, measurements of albumin, alpha-1 antitrypsin (AAT), alkaline phosphatase (ALP), alanine transaminase (ALT), aspartate transaminase (AST), gamma-glutamyl transpeptidase (GGT), prothrombin time, also expressed as international normalized ratio (INR), serum bilirubin, and urine bilirubin. Interestingly, CRP is the most reliable indicator of total body inflammation and is more reliable at predicts cardiac risk, if elevated, and more reliable predictor than cholesterol levels.

In order to perform the testing, subjects are screened and diagnosed with chronic sinusitis and then skin tested using the procedures taught herein.

### Example 2. Use and Synergy of Itraconazole and Ketoconazole in Killing Fungal Survival Spores

The vast majority of the subjects tested in Example 1 showed a delayed hypersensitivity to at least Candida fungal species and/or Aspergillus fungal species. The most predominant reaction included a delayed hypersensitivity to a Candida species including, but not limited to, Candida albicans and/or an Aspergillus species including, but not limited to, Aspergillus niger. Of the antifungals tested, a combination of itraconazole and ketoconazole showed the most striking effect at treating recurrent/chronic sinusitis, or at least inhibiting, a recurrence of chronic sinusitis. While not intending to be bound by any theory or mechanism of action, data and observations suggest that this is because the formulations, including the combinations of antifungals taught herein, were surprisingly effective together at killing, and/or at least inhibiting or preventing the replication of, the fungal survival spores and/or replicating fungi. Although each was effective against it's own target fungi individually, there appeared to be a significantly greater than additive effect when the two were used together. This combination was chosen due to its effectiveness at treatments, its resistance to systemic absorption by the tissue of the subject after topical administration, and negligible any side effects to the subject.

### Example 3. Evaluation of the Synergistic Activity of 2 Antifungal Agents (Itraconazole and Ketoconazole) with an Antibiotic (Vancomycin or Tobramycin or Amikacin) Against the Spores of 2 Fungi; Aspergillus Fumigatus and Candida albicans, Individually as Well as in Co-Cultivation (Aspergillus Fumigatus+Candida albicans)

This study evaluated the synergistic activity of 2 antifungal agents (Itraconazole and Ketoconazole) with an antibiotic (Vancomycin or Tobramycin or Amikacin) against the spores of 2 fungi; Aspergillus fumigatus MYA3626 and Candida albicans ATCC 90028; individually as well as in co-cultivation (Aspergillus fumigatus+Candida albicans). Synergy testing was carried out as per checkerboard assay by Victor Lorian methodology. See, for example, Lorian, V. Antibiotics in Laboratory Medicine. Edition 5: (2005).

To perform the checkerboard assay, the following was performed:

Broth Microdilution Minimal Inhibitory Concentration (MIC) assays were performed to obtain the MIC values for Itraconazole and Ketoconazole with an antibiotic (Vancomycin or Tobramycin or Amikacin) against the two fungal strains.

The in vitro activity of Itraconazole+Ketoconazole combination was determined against the 2 fungi; Aspergillus fumigatus MYA3626 and Candida albicans ATCC 90028; individually as well as in co-cultivation (Aspergillus fumigatus+Candida albicans).

Materials and Methods

Fungal Strains:

Aspergillus fumigatus MYA3626 and Candida albicans ATCC 90028 strains were obtained from from the American Type Culture Collection (Manassas, Va.). The strains were maintained as a frozen glycerol stocks at −80° C. The fungal strains were prepared by thawing a glycerol stock, streaking and growing onto Sabouraud's Dextrose Agar (SDA) plates, overnight at 35±2° C. in aerobic conditions and used for the assay.

TABLE 1
Details of fungi strains.

| # | Organism | Isolate # | Phenotype |
|---|---|---|---|
| 1 | A. fumigatus | MYA3626 | QC |
| 2 | C. albicans | ATCC 90028 | QC |
| 3 | Co-cultivation of A. fumigatus + | MYA3626 + | QC |
| | C. albicans | ATCC 90028 | |

Antifungal agents: Itraconazole (Cat #16657, Lot #087K1322), Ketoconazole (Cat # K1003, Lot # SLBR1290V). Stocks were prepared in appropriate solvents following the CLSI guidelines. See, for example, Reference method for broth dilution antifungal susceptibility testing of filamentous fungi, approved standard, 2nd ed. CLSI document M38-A2, vol. 28. CLSI, Wayne, Pa. (2008).

Inoculum Preparation of Fungal Spores:

The completely synthetic medium RPMI-1640 (with glutamine, without bicarbonate, and with phenol red as a pH indicator) was buffered with 0.165 mol/L MOPS (3-[N-morpholino] propanesulfonic acid) buffer was used as the growth medium for the fungal strains. Overnight cultures of the fungal strains were grown on Difco™ Sabouraud's Dextrose Agar (SDA) plates at 35° C. To induce conidium and sporangiospore formation, the culture plates were transferred to incubator and grown for 5-7 days 30° C. or until good sporulation was obtained. Approximately, 10 mL of 0.85% saline containing 0.01% of Tween 80 was used to poured on to the agar plate containing conidia or sporangiospores and hyphal fragments. The suspension containing conidia or sporangiospores and hyphal fragments was aspirated and transferred to a sterile tube. This mixture was filtered to separate the spores from the vegetative cells of the fungi. The supernatant containing only fungal spores was used for all the assays. Fungal spore inoculum was prepared in RPMI-1640 (with glutamine, without bicarbonate, and with phenol red as a pH indicator) was buffered with 0.165 mol/L MOPS (3-[N-morpholino] propanesulfonic acid) buffer to a final density of 106 CFU/mL using a hemocytometer.

Minimal Inhibitory Concentration (MIC) Testing:

Each MIC assay included 11 2-fold serial dilutions of the antifungal agents. Test concentrations for the test articles were tested at the following concentrations: 64 μg/mL, 32 μg/mL, 16 μg/mL, 8 μg/mL, 4 μg/mL, 2 μg/mL, 1 μg/mL, 0.5 μg/mL, 0.25 μg/mL, 0.125 μg/mL, and 0.0625 μg/mL. Broth microdilution assays were performed according to the procedures detailed in CLSI document M38-A2 (CLSI, 2008). Briefly, 2× stock solutions of test articles were prepared at 128 μg/mL. A two-fold serial dilution of the stock solution were performed. Diluted stock solutions of the fungal spores were adjusted (to be equivalent to 10⁶ CFU/mL). 1× final concentration of the antifungals was obtained when they were combined with the fungal spore broth in the microtiter plate. Plates were incubated for 3-5 days at 35±2° C. in an ambient air incubator. MIC values recorded are the minimum concentration of the test articles that inhibits growth as observed by unaided eye. The MIC values was expressed in μg/mL. The assay was carried out in duplicates.

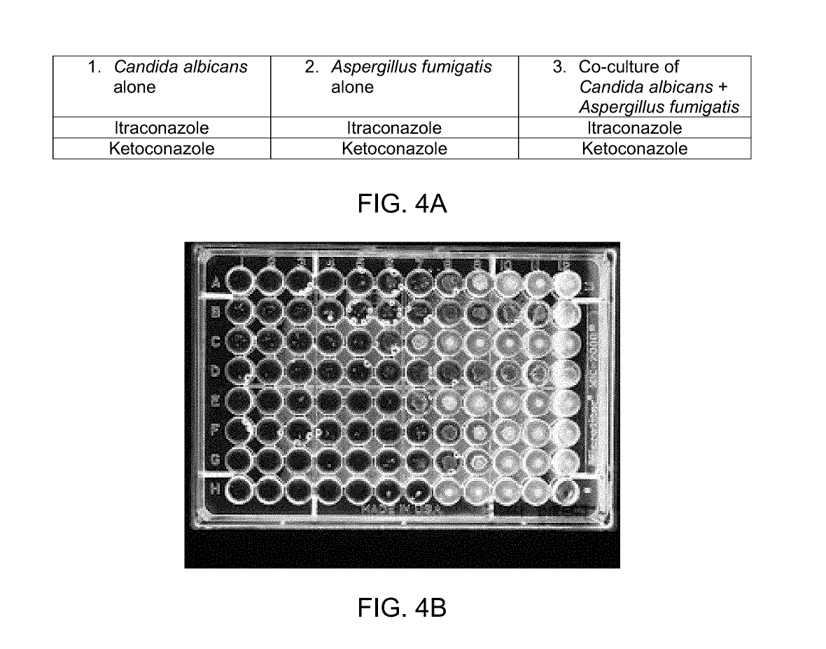

FIGS. 4A and 4B illustrate the components of an assay tray, and the assay tray, used in an MIC assay to identify component synergy in embodiments taught herein, according to some embodiments. FIG. 4A shows the fungi, alone and in combination, that were used as the test articles, as well as the antifungals that were applied to the test articles alone and in combination. FIG. 4B shows the assay tray configuration. Once the MICs were determined, they were used for the synergy testing.

Synergy Measurement by Checkerboard Assay:

In vitro activity of the test articles were tested in combination by the Checkerboard assay. See, for example, Lorian, V. Antibiotics in Laboratory Medicine. Edition 5: (2005). The assay was carried out in duplicates. The starting concentrations for the test articles was determined as per the MIC data. Two-fold serial dilutions of 8×MIC for the antifungal agents; Itraconazole and Ketoconazole was performed separately. When Itraconazole and Ketoconazole were combined in a microtiter plate, the concentration of both agents was 2×MIC. 1× final concentration of the antifungal agents were obtained when they were combined with the 10⁶ CFU/mL fungal spore inoculum in the microtiter plate. The plates were incubated at 35° C. for 3-5 days. The assay plates were visually inspected to confirm the MIC endpoint and used to calculate the Fractional Inhibitory Concentration (FIC) Index to determine any synergistic interaction.

The FIC Index was calculated as follows:

FIC_{A}=A/MIC_{A};

FIC_{B}=B/MIC; and,

FIC Index=FIC_{A}+FIC_{B}
where, A and B are the MIC of the test article and an antifungal in combination (in a single well). MIC_{A} and MIC_{B} are the MICs of each antifungal individually.

FIC Index value was used to categorize the interaction, as follows:

TABLE 2

| | FIC Index |
|---|---|
| Synergy | ≦0.5 |
| Antagonism | >4 |
| Additive or indifference | 0.51-4 |

Co-Culture Synergy Assays:

A co-culture of spores of Aspergillus fumigatus and Candida albicans were combined in 1:1 ratio and grown in RPMI-1640 (with glutamine, without bicarbonate, and with phenol red as a pH indicator) was buffered with 0.165 mol/L MOPS (3-[N-morpholino] propanesulfonic acid) buffer to a final density of 10⁶ CFU/mL using a hemocytometer.

Determination of MIC Values:

The following concentrations were used for MIC Assay for the antifungal test articles: 64 μg/mL, 32 μg/mL, 16 μg/mL, 8 μg/mL, 4 μg/mL, 2 μg/mL, 1 μg/mL, 0.5 μg/mL, 0.25 μg/mL, 0.125 μg/mL, and 0.0625 μg/mL. The MIC data is shown in Table 3.

Table 3 shows that Itraconazole had better MIC values than Ketoconazole against Aspergillus fumigatus alone, Candida albicans alone, and against Aspergillus fumigatus+Candida albicans co-culture. Itraconazole and Ketoconazole had MIC values of 0.5 μg/mL and 16 μg/mL against A. fumigatus, respectively, and a MIC value of 0.06 μg/mL against C. albicans. For the co-culture of Aspergillus fumigatus+Candida albicans, Itraconazole had a MIC value of 0.5 μg/mL while Ketoconazole had a MIC value of 16 μg/mL.

TABLE 3

| | MIC of Itraconazole | MIC of Ketoconazole |
|---|---|---|
| Strains | (μg/mL) | (μg/mL) |
| Aspergillus fumigatus | 0.5 | 16 |
| Candida albicans | 0.06 | 0.06 |
| Aspergillus fumigatus + | 0.5 | 16 |
| Candida albicans | | |

Determination of Optimal Itraconazole and Ketoconazole Combination & Concentration Using Synergy Assay:

To determine if Itraconazole and Ketoconazole combination had synergistic antifungal activity, a synergy assay was carried out with Itraconazole (1 μg/mL, 0.5 μg/mL, 0.25 μg/mL, 0.125 μg/mL, 0.0625 μg/mL, 0.0312 μg/mL, and 0.0156 μg/mL) and Ketoconazole (8 μg/mL, 4 μg/mL, 2 μg/mL, 1 μg/mL, 0.5 μg/mL, 0.25 μg/mL, 0.125 μg/mL, 0.0625 μg/mL, 0.0312 μg/mL, 0.0156 μg/mL and 0.0078 μg/mL) against Aspergillus fumigatus alone, Candida albicans alone, and against Aspergillus fumigatus+Candida albicans co-culture.

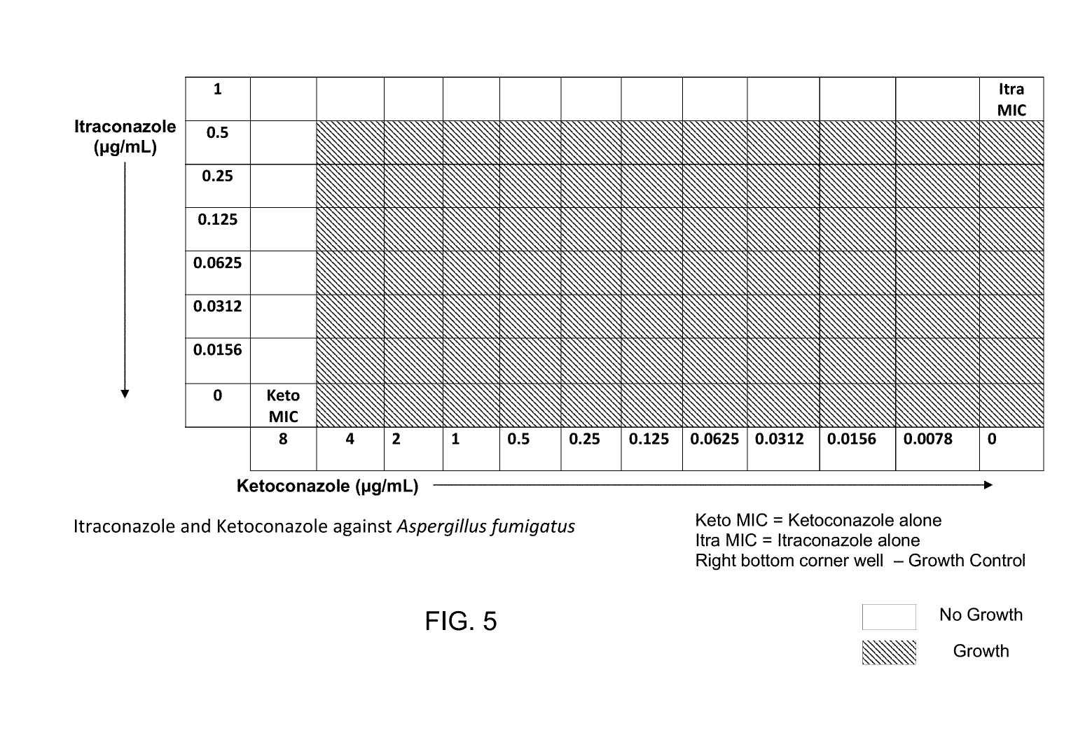

FIG. 5 shows the synergy data for Itraconazole and Ketoconazole against Aspergillus fumigatus, according to some embodiments. The starting concentrations range for Itraconazole were 1 μg/mL to 0.0156 μg/mL and for Ketoconazole were 8 μg/mL to 0.0078 μg/mL. As can be seen in FIG. 5, the MIC for Ketonconazole was greater than 4 μg/mL, and the MIC for Itraconazole was greater than 0.5 μg/mL, for Aspergillus fumigatus.

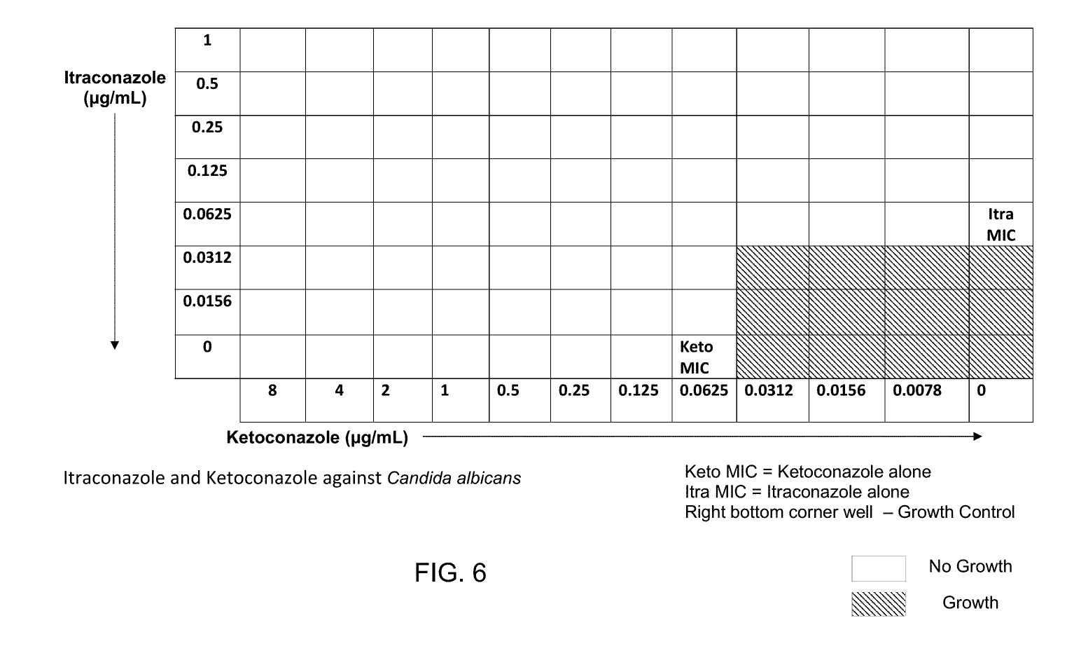

FIG. 6 shows the synergy data for Itraconazole and Ketoconazole against Candida albicans, according to some embodiments. The starting concentration range for Itraconazole was 1 μg/mL to 0.0156 μg/mL and for Ketoconazole were 8 μg/mL to 0.0078 μg/mL. As can be seen in FIG. 6, the MIC for Ketonconazole was greater than 0.0312 μg/mL, and the MIC for Itraconazole was greater than 0.0312 μg/mL, for Candida albicans.

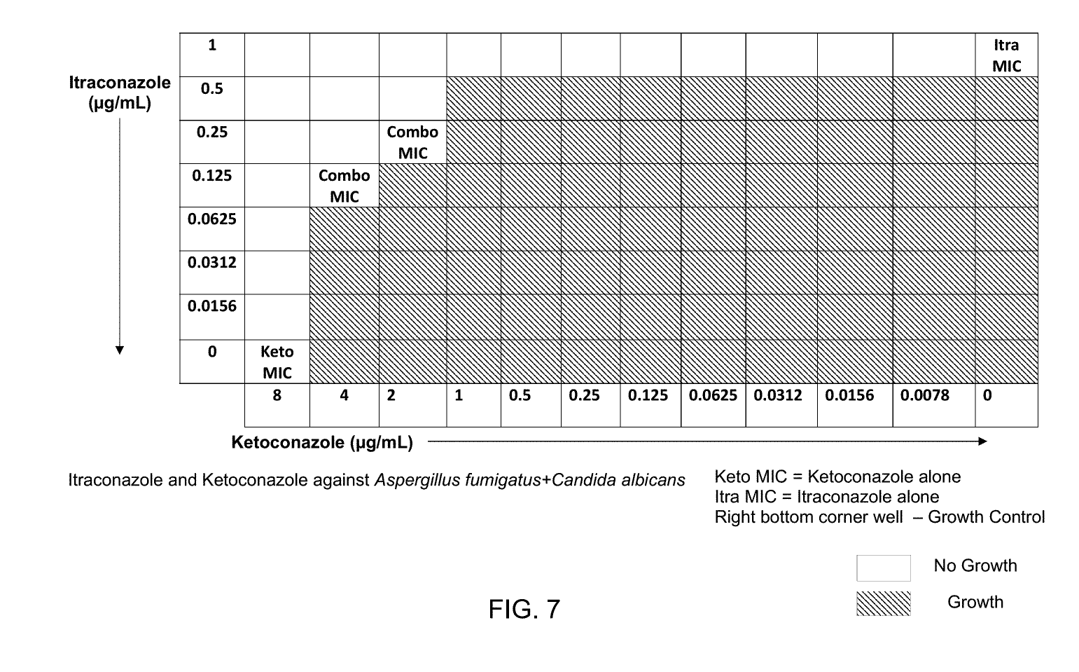

FIG. 7 shows the synergy data Itraconazole and Ketoconazole against Aspergillus fumigatus+Candida albicans, according to some embodiments. The starting concentrations range for Itraconazole were 1 to 0.0156 μg/mL and for Ketoconazole were 8 to 0.0078 μg/mL. As can be seen in FIG. 7, (i) the MIC for Ketonconazole alone was greater than 4 μg/mL, and the MIC for Itraconazole alone was greater than 0.5 μg/mL, for the combination of Aspergillus fumigatus and Candida albicans. However, (ii) the MIC for a combination of Ketoconazole with Itraconazole is expressed as be greater than 0.0625 μg/mL Ketonazaone with greater than 0.125 μg/mL Itraconazole, and (iii) greater than 0.125 μg/mL Itraconazole with greater than 1 μg/mL Ketoconazole. As such, the antifungals worked better in the combination than each did alone.

Table 4 repeated the MIC study and again shows that Itraconazole had better MIC values than Ketoconazole against Aspergillus fumigatus alone, Candida albicans alone, and against Aspergillus fumigatus+Candida albicans co-culture. This time, an acceptable variation from Table 3 was shown. Itraconazole and Ketoconazole had MIC values of 1.0 μg/mL and 8 μg/mL against A. fumigatus, respectively, and again a MIC value of 0.06 μg/mL against C. albicans. For the co-culture of Aspergillus fumigatus+Candida albicans, Itraconazole had a MIC value of 1.0 μg/mL while Ketoconazole had a MIC value of 8 μg/mL. is a summary of the synergy assays of Itraconazole+Ketoconazole combination against Aspergillus fumigatus alone, Candida albicans alone and co-culture of Aspergillus fumigatus+Candida albicans. It can be seen that the MIC values for Ketoconazole were again decreased, when the two anti-fungal agents, Itraconazole and Ketoconazole, were used in combination in comparison to their individual MIC values. The FIC Index values of two anti-fungal agents, Itraconazole and Ketoconazole, provided values that were clearly synergistic in the case of the co-culture of Aspergillus fumigatus+Candida albicans. For example, it can be seen that the 0.25 μg/mL Itra and 2 μg/mL Keto (ratio of 8:1 Keto/Itra) had a synergistic effect in case of the co-culture of Aspergillus fumigatus+Candida albicans. One of skill will appreciate that the variation in MIC values is within the expected range.

TABLE 4

| | | | MIC of |
|---|---|---|---|
| | MIC of | MIC of | Itrakconazole + |
| | Itraconazole | Ketoconazole | Ketoconazole |
| Strains | (μg/mL) | (μg/mL) | (μg/mL) |
| Aspergillus fumigatus | 1.0 | 8 | |
| Candida albicans | 0.06 | 0.06 | |
| Aspergillus fumigatus + | 1.0 | 8 | 0.125 Itra and |
| Candida albicans | | | 4 Keto*; and |
| | | | 0.25 Itra and |
| | | | 2 Keto** |

*FIC of 0.63 which suggests synergy in the combination
**FIC of 0.5 which is proof of synergy in the combination

The Itraconazole+Ketoconazole Combination Shows a Synergistic Effect.

Another way of expressing this is that the MIC values of both Itraconazole and Ketoconazole were effectively reduced when used together. As shown above, for example, the MIC of Itraconazone was reduced from 1 to 0.25 Aspergillus fumigatus and Candida albicans and Ketoconazole was reduced from 8 to 2 μg/mL in the data shown above. These results are surprising and valuable to those of skill in the art, for at least the reason that the synergy in the combination of Itraconazole+Ketoconazole combination decreases the MIC of Itraconazole or Ketoconazole by at least 4-fold and hence, ultimately, its possible to decrease the dosage, as well as any potential side-effects associated, in the administration of Itraconazole or Ketoconazole in clinics.

As illustrated by FIG. 7, one of skill will appreciate that any combination of greater than about 0.5 μg/mL Itraconazole with greater than 0 μg/mL of Ketoconazole, or any combination of greater than about 4 μg/mL Ketoconazole and greater than 0 μg/mL Itraconazole, would be expected to at least inhibit a co-culture of the test article which was shown as having a final density of 10⁶ CFU/mL of about 1:1 ratio of Aspergillus fumigatus and Candida albicans as determined using a hemocytometer (“the test article”). Specifically, a combination of greater than about 0.5 μg/mL Itraconazole with at least 0.0078 μg/mL of Ketoconazole (one of skill will appreciate that a ratio of 64:1 Itraconazole/Ketoconazole is a concrete boundary, although the ratio can be higher), or any combination of greater than about 4 μg/mL Ketoconazole and at least about 0.0156 μg/mL Itraconazole (one of skill will appreciate that a ratio of 256:1 Ketoconazole/Itraconazole is a concrete boundary, although the ratio can be higher).

Moreover, although these other ratios were operable, the stair-step portion of FIG. 7 is evidence of synergy.

Upon further analysis, it was seen that the growth of a co-culture of Aspergillus fumigatus and Candida albicans can be at least inhibited using combinations of the antifungals in ratios ranging from (i) a ratio of greater than at least about 0.0625 μg/mL Itraconazole to greater than at least about 2.0 μg/mL Ketoconazole (about 32:1 Ketoconazole/Itraconazole); (ii) a ratio of greater than at least about 0.125 μg/mL Itraconazole to greater than at least about 1.0 μg/mL Ketoconazole (8:1 Ketoconazole/Itraconazole).

### Example 4. Evaluation of the Synergistic Activity of Antifungal Agent Combinations

The synergy assay can also be performed using minimum inhibitory concentration (MIC) and minimum fungicidal concentration (MFC) to determine the presence of synergy in the combination of any antifungal combination taught herein on any fungus or combination of fungi taught herein. For example, itraconazole and ketoconazole can be further investigated as follows:
The activity of itraconazole can be tested on additional Aspergillus species including, for example, Aspergillus fumigatus, Aspergillus nigra (niger), and Aspergillus flavus, and combinations thereof;
The activity of ketoconazole can be tested on Candida species, such as Candida albicans, Candida tropicalis, Candida glabrata, and Candida parapsilosis, and combinations thereof; and,
Any combination of the any of the above Candida species and the Aspergillus species can then be tested for synergy.

First, the Candida species is tested alone against each of itraconazole and ketoconazole alone. Next, the Aspergillus species is tested alone against each of itraconazole and ketoconazole alone. And, finally, a co-culture of the Candida species and the Aspergillus species is tested against each of itraconazole and ketoconazole. The MFC values for each of itraconazole alone are used to compare activities for the synergy testing. A peptone glucose fluconazole agar is used as selective media to differentiate Aspergillus from Candida.

The co-culture of the Candida and Aspergillus species can be grown using brain-heart infusion broth, and a checkerboard synergy format will be used. MIC values will be measured, and synergy will be evaluated. The synergy concentration will be plated for colony forming unit (CFU) counts on sabouraud dextrose agar (SDA) plates supplemented with ampicillin (0.008 mg/ml) and erythromycin (0.075 mg/ml) to prevent growth of a non-targeted microbe. Peptone glucose fluconazole agar will be used as a selective media to differentiate Aspergillus from Candida.

### Example 5. Use and Synergy of Itraconazole and Ketoconazole in Killing Fungal Survival Spores with the Addition of Tobramycin or Vancomycin

The methods taught herein can also be used to determine activity and synergy of the antifungals alone and in combination with any antibiotic or combination of antibiotics taught herein.

Tobramycin and/or vancomycin are used in some embodiments, for example. Tobramycin is usually given intravenously or by intramuscular injection, as it is very, very poorly absorbed orally and is not absorbed systemically when used topically. As such, these characteristics mitigate, and likely eliminate, risk of side effects when administered topically. Moreover, Tobramycin has excellent activity against both gram negative and gram positive organisms, and most patients are sensitive to tobramycin. Moreover, it was also discovered that patients that have chronic rhinosinusitis commonly have fungal infections that are concomitant with infections with gram negative organisms, such as Proteus sp, Pseudomonas sp, E. Coli, and Serratia sp. In some embodiments, the gram negative bacteria can include Escherischia coli, Pseudomonas aeruginosa, Serratia marescens, Klebsiella species, Citrobacter species, and Proteus mirabilis.

Without intending to be bound by any theory or mechanism of action, it appears that these gram-negative organisms are existing in a symbiotic relationship with the fungi. As such, the antibiotics are selected to eliminate the symbiotic relationship between the bacterial and the fungi to reduce the chance of recurrence of the chronic rhinosinusitis. Vancomycin may be preferred, for example, when the patient is known to have a methicillin-resistant Staphylococcus aureus (MRSA) infection that accompanies the fungal infection in the chronic sinusitis.

In addition to the methods taught above, the co-culture of the Candida and Aspergillus species can be grown using brain-heart infusion broth. A combination of test antibiotic (vancomycin/tobramycin) and the most-effective antifungal combination (ketoconazole and itraconazole) will be added in the checkerboard synergy format. MIC values will be measured, and synergy will be evaluated. The synergy concentration will be plated for colony forming unit (CFU) counts on sabouraud dextrose agar (SDA) plates supplemented with ampicillin (0.008 mg/ml) and erythromycin (0.075 mg/ml) to prevent growth of a non-targeted microbe. Peptone glucose fluconazole agar will be used as a selective media to differentiate Aspergillus from Candida.

### Example 6. Addition of Mometasone to Control Inflammation and Access to the Ostiomeatal Complex in the Treatment

Mometasone furoate was then added to the combination of antifungals and antibiotic, as it is an extremely potent topical anti-inflammatory which is easily delivered into the nasal cavity and into the sinuses. It has very little systemic absorption and is very safe, as it is approved for children under the age of 3. The anti-inflammatory helps reduce the inflammation that would arise affect the ability to get the antifungals and antibiotic to the ostiomeatal complex. See, for example, FIGS. 2A-2D for reference to how access to the ostiomeatal complex can be affected by swelling. Since the downside of using the anti-inflammatory is the possibility of getting a secondary fungal infection, which makes the combination of antifungal agents even more desired in the treatment methods taught herein.

### Example 7. A 4-Component Formulation and Administration Protocol

The examples provided herein show the research and development that uncovered a 4-component formulation that appears to provide the best results overall for in the treatment of chronic rhinosinusitis. The 4-component formulation includes (i) a first antifungal; (ii) a second antifungal that complements the first antifungal; (iii) an antibiotic that complements the combination of antifungals by killing gram-negative bacteria that appear to be symbiotic with the fungi present in the infection, and (iv) a potent anti-inflammatory that helps reduce the inflammation that would arise affect the ability to get the antifungals and antibiotic to the ostiomeatal complex.

A formulation can be made, for example, to have from about 40 mg to about 375 mg, and preferably about 125 mg tobramycin or about 160 mg vancomycin; from about 13 mg to about 120 mg, and preferably about 40 mg, of itraconazole; from about 13 mg to about 120 mg, and preferably about 40 mg, of ketoconazole; and, from about 0.2 mg to about 5.4 mg, and preferably from about 0.6 mg to about 1.8 mg, of mometasone furoate. The formulation can be topically administered intranasally to the ostiomeatal complex of a subject twice daily for an first administration period of at least about one week, two weeks, three weeks, or four weeks. In some embodiments, the first administration period can be stopped for a rest period of about 1 day, 2 days, 3 days, 4 days, 5 days, 6 days, 7 days, 8 days, 9 days, 10 days, 11 days, 12 days, 13 days, or 14 days, and then resumed at once per day for at least an additional about one week, two weeks, three weeks, or four weeks. The dosage forms can be powder or capsule, for example, and can be delivered to an ostiomeatal complex using an aqueous carrier having any volume taught herein, such as 1-10 ml total volume, 2-5 ml total volume, or perhaps 3 ml total volume, for single topical administrations 1-3× daily, using administration schedules taught herein, for example. In some embodiments, the total volume of the formulation can be 1-100 ml, 1-90 ml, 1-80 ml, 1-70 ml, 1-60 ml, 1-50 ml, 1-40 ml, 1-30 ml, 1-20 ml, 1-10 ml, 1-5 ml, or any volume or range therein in increments of 1 ml. As such, the administered volume, which can be the amount of carrier per dose, can be 1 ml, 2 ml, 3 ml, 4 ml, 5 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, or 100 ml, or any amount or range therein in increments of 0.5 ml; and, perhaps even more, as needed. In some embodiments, for example, the volume of aqueous carrier per dose can be any volume required to the desired amount of the formulation components to the target tissue site, such as the osteomeatal complex.

In some embodiments, the formulation has about 125 mg tobramycin, about 40 mg itraconazole, about 40 mg ketoconazole, and about 0.6 mg mometasone furoate. And, in some embodiments, the formulation has about 160 mg vancomycin, about 40 mg itraconazole, about 40 mg ketoconazole, and about 0.6 mg mometasone furoate.

Dosages in the form of capsules were made as follows, based on a 1:1 antifungal combination, and accompanied by an antibiotic and anti-inflammatory, as desired:

TABLE 5
125 mg tobramycin, 40 mg itraconazole, 40 mg ketoconazole,
0.6 mg mometasone in a xylitol capsule. Tobramycin reaches
the target amount when adjusted for water content.

| | | AMOUNT PER |
|---|---|---|
| | AMOUNT | CAPSULE (mg); not |
| COMPONENT | MIXED (g) | adjusted for water |
| tobramycin sulfate (powder) | 55.921 | 186 |
| Itraconazole | 12.006 | 40 |
| ketoconazole | 12.002 | 40 |
| mometasone furoate | 0.180 | 0.6 |
| Xylitol 3% polox base | 69.467 | 231 |

300 capsules were made with this formulation using a Jaansen capsule machine per SOP 2.3 and bottled, stored at room temperature. The capsules had a smooth cream color.

### Example 8. Case Studies

In all of the following studies, the skin testing of Example 1 was used to identify the inhalant antigen or antigens responsible for triggering systemic symptoms and the localized sinus symptoms. Inflammation is a shared symptom in many embodiments.

In some embodiments, for example, the chronic rhinosinusitis of the patients was all successfully treated. Moreover, the patients also experienced resolution of other symptoms related to other conditions including, for example, systemic inflammatory symptoms.

Methods

Each of the patients avoided additional exposure to the fungal spores that were identified by the skin test. The patients ostia was dilated, and the formulation was administered with a nebulizer. Additionally, systemic anti-fungal medication directed toward the specific fungi was administered for 4-6 weeks, followed by intermittent nebulization with mometasone and 2% mupirocin powder, which has both anti-fungal and anti-bacterial properties. These treatments were administered every other day to every third day for several months with C-reactive protein (CRP} levels checked every 4-6 weeks to monitor change.

Patient 1

Patient 1 is a 60 year old female, retired nurse who presented with chronic fatigue, chronic headaches, facial pain, and a sleep disorder characterized by Rapid Eye Movement (REM) sleep deprivation. The patient was a two pack/day smoker. The diagnostic skin test revealed moderate and severe delayed hypersensitivity reactions to Rhizopus, Fusarium, Candida, and Aspergillus. The patient was treated with a formulation of 125 mg tobramycin.

40 mg of itraconazole, 40 mg of ketoconazole, 0.6-1.8 mg of mometasone furoate administered by nebulizer twice daily for three weeks. Administration was stopped for one week and then resumed daily for three weeks. Following the treatment protocol, the patient's symptoms completely resolved.

Patient 2

Patient 2 is a 65 year old male who presented with severe fatigue, Parkinson's disease (precluding his ability to drive), severe tremors (precluding any meaningful use of his hands), and an inability to walk requiring spousal assistance, a walker, or a wheelchair. The patient's diagnostic skin test revealed delayed hypersensitivity reactions to Candida and Aspergillus. The patient was treated as mentioned in the Patient 1 profile, but has continued daily/every other day nebulization treatments for the last two years at his insistence. His symptoms improved such that he is now able to speak coherently, use his hands, walk without any assistance and drive his car again. The patient is now maintained on mometasone and powdered mupirocin nebulization.

Patient 3

Patient 3 is a 66 year old male and the owner of a lumber business who presented with a sleep disorder, fatigue, and severe somnolence. The patient also suffered from diffuse arthritis (physical exam consistent with rheumatoid arthritis), hypothyroidism, chronic prostatitis, facial pain, headaches and was status post coronary artery bypass surgery. The patient's diagnostic skin test revealed delayed hypersensitivity reactions to Candida and Aspergillus (severe). Upon reviewing the test results with the patient, noting the severe Aspergillus reaction, the fact that was raised that Aspergillus is used for fermentation of “green” marijuana leaves and production of THC, at which time the patient volunteered that he had smoked marijuana almost daily for more than 40 years. Once again, guided by identification of the offending antigen and its source, the treatment regimen was initiated utilizing nebulized itraconazole and mometasone, oral terbinafine, and an in-office balloon sinuplasty. At only six weeks post-treatment, the patient experienced resolution of his sleep disorder, facial pain and headaches and a reduction in arthritis signs and symptoms. The patient continues nasal nebulization for two weeks per month.

Patient 4

Patient 4 is a 54 year old male and retired software developer who presented with severe fatigue, fibromyalgia, Crohn's disease (being treated with Humira), a history of severe cardiovascular disease requiring bypass surgery, a sleep disorder, and evidence of severe fungal dermatosis of hands and feet as well as an elevated CRP level of 12 mg/L (high risk >3.0 mg/L), a predictor of high risk of coronary artery disease. The patient's diagnostic skin test revealed delayed hypersensitivity reactions to Candida and Aspergillus, and he was initially treated with nebulized itraconazole, ketaconazole and mometasone and balloon sinuplasty (now maintained on nebulized mometasone and mupirocin). At approximately two years post-initial treatment, the patient has no fibromyalgia, arthritis, his Crohn's disease has resolved (no longer taking Humira), and his hand and foot fungal infection has resolved. Most remarkably, a Computerized Tomography (CT) dye study showed absence of CAD, and the latest CRP is 0.5 mg/L (low risk <1.0 mg/L).

Patient 5

Patient 5 is a 60 year old male and clinical psychologist who developed severe fatigue, crescendo snoring, a sleep disorder, hypothyroidism, arthritis symptoms, gastroesophageal reflux disease, atrial fibrillation and three episodes of life threatening pericarditis after receiving allergy shots for 6-9 months. The allergy shots were to treat fungi spore antigens based on a positive histamine reaction to standard SET allergy testing. After determining the antigen content of the ‘allergy’ injections the patient was receiving on a bi-weekly basis, a diagnostic skin test was performed to try to uncover the delayed hypersensitivity reaction. The test revealed a severe delayed hypersensitivity reaction to the fungal spore antigens which had also elicited the positive histamine response to the SET testing. Providing treatment based only on the histamine reaction had set off a firestorm of symptoms and diseases as the patient had severe delayed hypersensitivity to the offending antigen. The treatment approach included discontinuing his allergy shots and initiating the treatment regimen described for Patient 1, based on the diagnostic skin test results. All symptoms and diseases have resolved since discontinuing the allergy shots and initiation of the treatment.

Patient 6

Patient 6 is a 53 year old male computer chip designer who received his diagnosis of rheumatoid arthritis eleven years ago. The patient has had increasing diffuse arthritic pain, weakness, and fatigue, while using the following medicines: (1) prednisone; (2) methotrexate; (3) hydroxychloroquine (an anti-parasitic medication used in patients with rheumatoid arthritis and or Lupus); and (4) sulfasalazine (Azulfidine) (an anti-inflammatory medicate often used to treat ulcerative colitis and rheumatoid arthritis). Despite the use of those medications, the patient's joint pain reached a level (by his own estimate) of 9 out of 10.

After a cognitive workup, including allergic testing, cultures and sinus CT scan plus taking of a careful history, it was determined that the patient suffered from chronic rhinosinusitis (CRS) and had a delayed hypersensitivity reaction to Candida albicans survival spore antigen. The patient underwent minimum invasive sinus surgery and nebulizer post-op. This treatment appeared to cure all of the patient's CRS symptoms and his sleep disorder, but his rheumatoid arthritis symptoms remained. The patient was seen again, and repeat cultures revealed persistent sinus, oral, and likely gastrointestinal Candidiasis. On this basis, a course of fluconazole, doxycycline, and clotrimazole, a restriction of dietary products fermented with yeast, and institution of the 4-drug nebulization was begun. In only nine days, this resulted in a decrease of joint pain to “3.5 out of 10”, the patient is off all his prior medicates and the patient reports an increase in muscle strength and general energy.

Patient 7

Patient 7 is a 39 year old female insurance executive who was originally referred by her family physician and insurer for severe fatigue, crescendo snoring, chronic facial pain, chronic post nasal drainage, sensation of throat “closed off”, choking at night, severe nasal obstruction, and reflux esophagitis in association with Zenker's Diverticulum of her esophagus. The patient also suffered from hypothyroidism and very significant weight gain over the last 5 years. The patient presented with a very high CRP level 1(2 mg/L) indicating very high system inflammation.

The patient underwent the standard workup for CRS, sleep disorder, and nasal obstruction, plus our diagnostic skin test and sinus CT scan. The results showed: a very positive CT scan consistent with CRS; positive delayed hypersensitivity reaction to Candida survival spore antigen; and culture revealed S. aureus, a gram negative bacteria, sensitive to tobramycin, and Candida albicans, sensitive to both itraconazole and ketoconazole.

The patient underwent a minimally invasive sinus procedure and was treated with the oral antifungal agents fluconazole, itraconazole, and terbinafine. The patient had improvement of all her symptoms (headache gone, improved sleep, increased energy, some improvement of sleep quality and nasal obstruction, some increased energy and some spontaneous weight loss). However, the patient did not demonstrate a robust improvement of all CRS and autoimmune disease related symptoms.

On follow-up, the patient was found to have persistent oral and sinus Candidiasis (infection with Candida albicans, to which the patient mounts a florid hypersensitive reaction). After once again treating the patient with oral medications for Candida, it was determined that the patient needed a revision sinus procedure (balloon sinuplasty) to widely open all sinuses and to facilitate the draining and reconciliation of the Candida Sinusitis. Although this in-office procedure had noticeable positive results, the patient still was not enjoying complete recovery. At this point, the patient began our nebulizer/sinus irrigation using the formulation described for Patient 1. After only ten days of the twice daily regimen, both the patient's physical exam and symptom set dramatically improved.

### Example 9. Effect of Treatment on Other Disorders, Including Autoimmune and Autoimmune-Mediated Disorders

The treatment of chronic rhinosinusitis is taught above. However, other conditions can be treated, at least indirectly, through the administration of the compositions and formulations as taught herein. For example, the compositions and formulations can be administered, as taught herein, to any human secretory tissue. In some embodiments, the administration of the compositions and formulations taught herein to the secretory tissue in the ostiomeatal complex, for example, can result in an effective treatment of Crohn's disease, rheumatoid arthritis, colitis, psoriasis, and ankylosing spondylitis, to name a few, with the goal of preventing, treating, inhibiting, and/or ameliorating the symptoms of any such condition. Other autoimmune, and autoimmune-related, diseases that can be treated using the methods taught herein include, but are not limited to Addison's disease, cardiomyopathy, celiac disease, Graves' disease, lupus, Meniere's disease, multiple sclerosis, myositis, and scleroderma. One of skill will appreciate that most any autoimmune, or autoimmune related, disorder can be treated using the methods taught herein.

Rheumatoid arthritis is a condition that be treated using the methods taught herein. Rheumatoid arthritis is a long-term autoimmune disorder that primarily affects joints. It typically results in warm, swollen, and painful joints, and the pain and stiffness often worsen following rest. Most commonly, the wrist and hands are involved, with the same joints typically involved on both sides of the body.

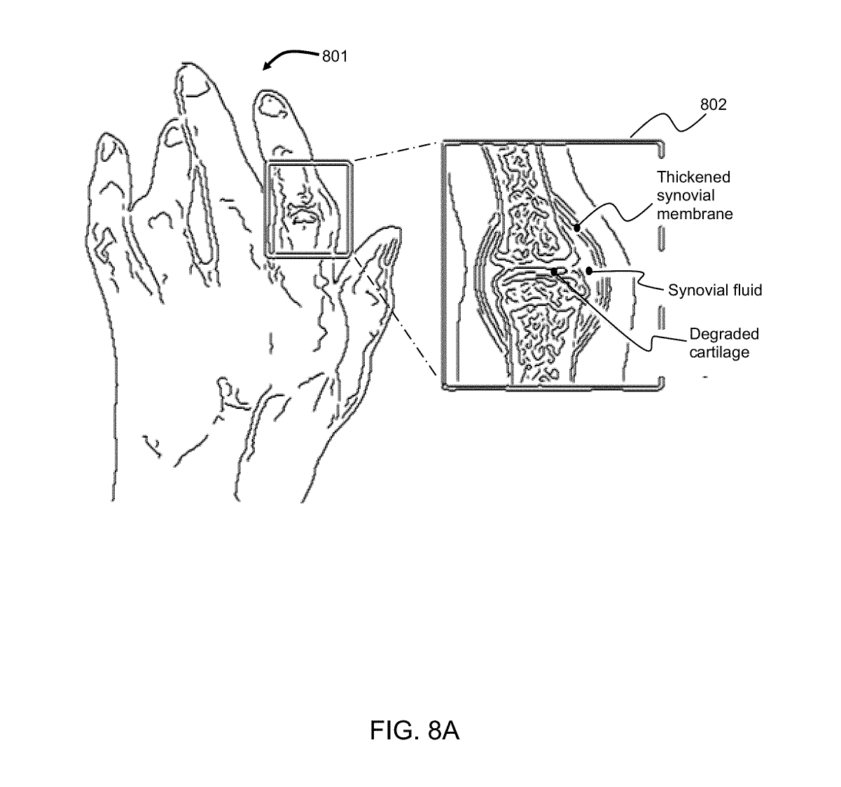

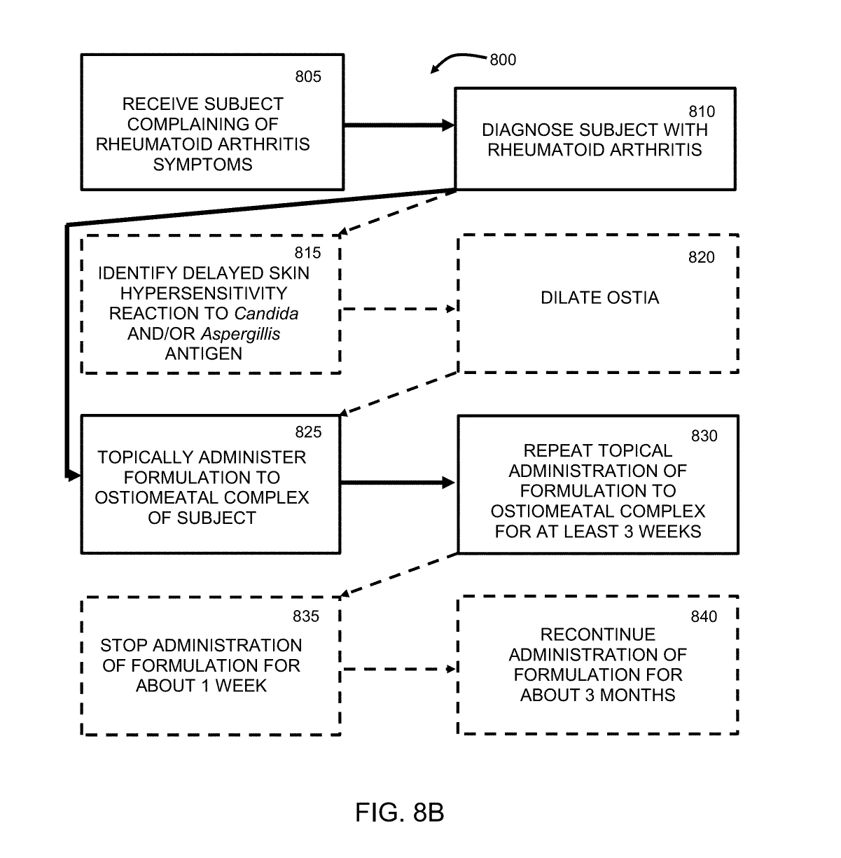

FIGS. 8A and 8B illustrate the use of the methods taught herein in the treatment of rheumatoid arthritis, according to some embodiments. FIG. 8A illustrates the hand of a subject 801 having rheumatoid arthritis, as well as the tissue changes in a proximal interphalangeal joint 802 of the index finger of the subject. The subject 801 is suffering the deleterious effects of rheumatoid arthritis that include, for example, a thickened synovial membrane surrounding synovial fluid that functions to lubricate the degraded cartilage.

In the method 800 of FIG. 8B, such treatment methods can include first receiving 805 a subject 801 that is complaining of rheumatoid arthritis symptoms and then diagnose 810 the subject with rheumatoid arthritis through methods that are well-known to the skilled artisan.

A topical administration of the combination of the antifungal agents can then be repeated 830 and, in some embodiments, repeated for at least 3 weeks. In these embodiments, the topically administering can include contacting a first antifungal agent with the ostiomeatal complex, the first antifungal selected as effective at killing Candida species of fungus, inhibiting the growth and/or reproduction of the Candida species, or a combination thereof; and, contacting a second antifungal agent with the ostiomeatal complex, the second antifungal selected as effective at killing an Aspergillus species of fungus, inhibiting the growth and/or reproduction of the Aspergillus species, or a combination thereof. In such embodiments, the topically administering of the first antifungal agent can be concurrent with the topically administering of the second antifungal agent. It should be appreciated that the agents, compositions, and formulations taught herein are administered to effectively kill the survival spores of each type of fungus taught herein in order to at least inhibit, and preferably prevent, the recurrence of the fungus, and thus rheumatoid arthritis, in the subject being treated.

In some embodiments, the method can further comprise topical administration of any one or any combination of corticosteroids to the subject. In some embodiments, the method can further comprise topical administration of any one or any combination of antibiotics to the subject. In some embodiments, the method can further comprise topical administration of any one or any combination of antibiotics and any one or any combination of corticosteriods to the subject.

Crohn's disease is a condition that be treated using the methods taught herein. Crohn's disease is a type of inflammatory bowel disease (IBD) that may affect any part of the gastrointestinal tract from mouth to anus. Signs and symptoms often include abdominal pain, diarrhea (which may be bloody if inflammation is severe), fever, and weight loss. Other complications may occur outside the gastrointestinal tract and include anemia, skin rashes, arthritis, inflammation of the eye, and feeling tired. The skin rashes may be due to infections as well as pyoderma gangrenosum or erythema nodosum. Bowel obstruction also commonly occurs and those with the disease are at greater risk of bowel cancer. It results in a chronic inflammatory disorder, in which the body's immune system attacks the gastrointestinal tract possibly directed at microbial antigens.

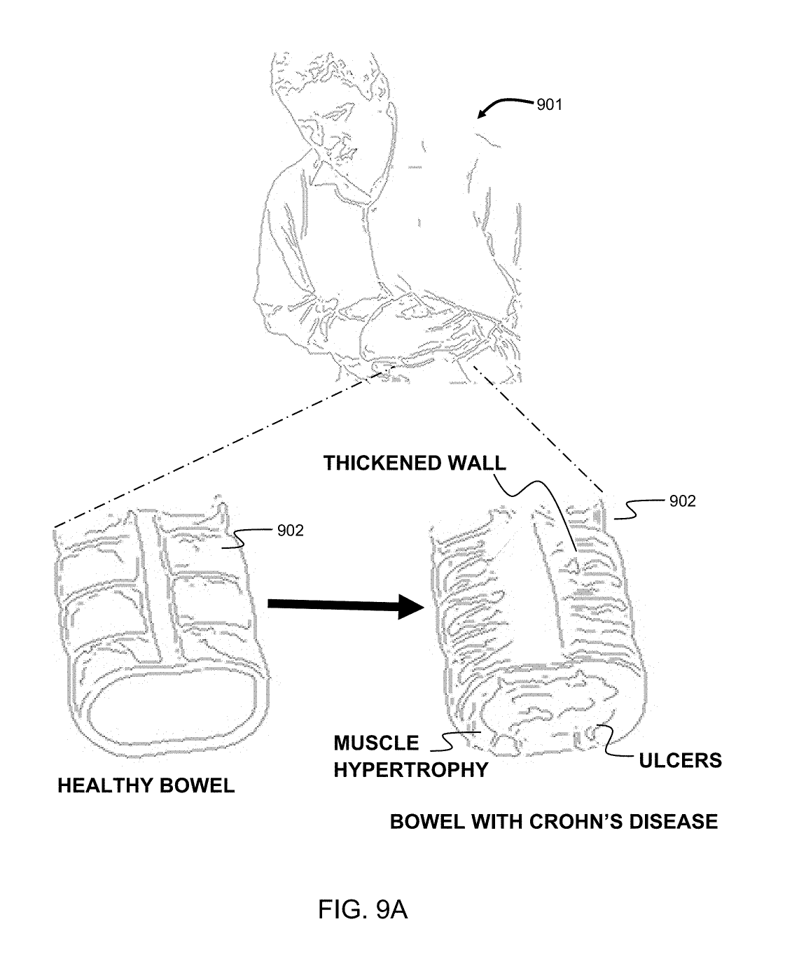

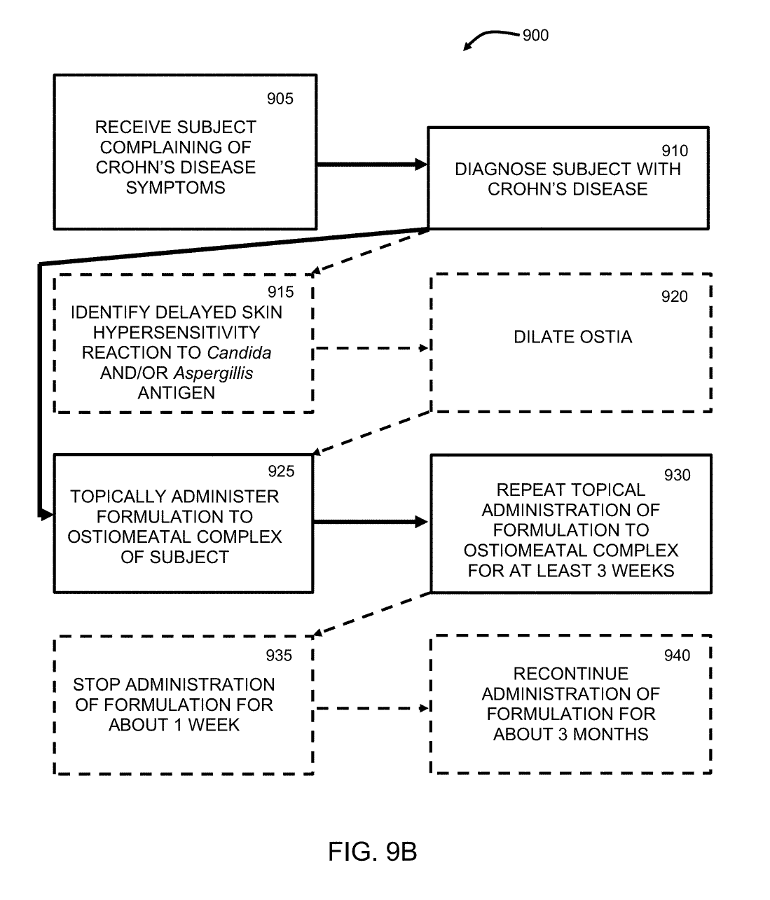

FIGS. 9A and 9B illustrate the use of the methods taught herein in the treatment of Crohn's disease, according to some embodiments. FIG. 9A illustrates a subject 901 having Crohn's, as well as the tissue changes in a bowel 902 of the subject 901. The subject 901 is suffering the deleterious effects of Crohn's that include, for example, sever abdominal pain, along with muscle hypertrophy and ulcers in the bowel.

In the method 900 of FIG. 9B, such treatment methods can include first receiving 905 a subject 901 that is complaining of Crohn's disease symptoms and then diagnose 910 the subject with Crohn's disease through methods that are well-known to the skilled artisan.

A topical administration of the combination of the antifungal agents can then be repeated 930 and, in some embodiments, repeated for at least 3 weeks. In these embodiments, the topically administering can include contacting a first antifungal agent with the ostiomeatal complex, the first antifungal selected as effective at killing Candida species of fungus, inhibiting the growth and/or reproduction of the Candida species, or a combination thereof; and, contacting a second antifungal agent with the ostiomeatal complex, the second antifungal selected as effective at killing an Aspergillus species of fungus, inhibiting the growth and/or reproduction of the Aspergillus species, or a combination thereof. In such embodiments, the topically administering of the first antifungal agent can be concurrent with the topically administering of the second antifungal agent. It should be appreciated that the agents, compositions, and formulations taught herein are administered to effectively kill the survival spores of each type of fungus taught herein in order to at least inhibit, and preferably prevent, the recurrence of the fungus, and thus the Crohn's disease, in the subject being treated.

In some embodiments, the method can further comprise topical administration of any one or any combination of corticosteroids to the subject. In some embodiments, the method can further comprise topical administration of any one or any combination of antibiotics to the subject. In some embodiments, the method can further comprise topical administration of any one or any combination of antibiotics and any one or any combination of corticosteriods to the subject.

Rosacea is a condition that be treated using the methods taught herein. Rosacea is a long-term skin condition that typically affects the face. It results in redness, pimples, swelling, and small and superficial dilated blood vessels. Often the nose, cheeks, and forehead, and chin are most involved. A red enlarged nose may occur in severe disease, a condition known as rhinophyma. Unfortunately, the cause of Rosacea is considered to be unknown to those skilled in the art and, as such, the condition is considered to be incurable at this time. Topical treatments are used to treat symptoms using, for example, metronidazole, doxycycline, or tetracycline. Other treatments that are sometimes used, and may show a benefit, include, for example, brimonidine cream, ivermectin cream, and isotretinoin; and, dermabrasion or laser surgery may also be used.

FIGS. 10A and 10B illustrate the use of the methods taught herein in the treatment of rosacea, according to some embodiments. FIG. 10A illustrates a subject 1001 having rosacea, as well as the tissue changes that occur on the skin of the subject 1001. The subject 1001 is suffering the deleterious effects of rosacea that include, for example, prominent blood vessels, along with the possible rhinophyma of the nose.

In the method 1000 of FIG. 10B, such treatment methods can include first receiving 1005 a subject 1001 that is complaining of rosacea symptoms and then diagnose 1010 the subject with rosacea through methods that are well-known to the skilled artisan.

A topical administration of the combination of the antifungal agents can then be repeated 1030 and, in some embodiments, repeated for at least 3 weeks. In these embodiments, the topically administering can include contacting a first antifungal agent with the ostiomeatal complex, the first antifungal selected as effective at killing Candida species of fungus, inhibiting the growth and/or reproduction of the Candida species, or a combination thereof; and, contacting a second antifungal agent with the ostiomeatal complex, the second antifungal selected as effective at killing an Aspergillus species of fungus, inhibiting the growth and/or reproduction of the Aspergillus species, or a combination thereof. In such embodiments, the topically administering of the first antifungal agent can be concurrent with the topically administering of the second antifungal agent. It should be appreciated that the agents, compositions, and formulations taught herein are administered to effectively kill the survival spores of each type of fungus taught herein in order to at least inhibit, and preferably prevent, the recurrence of the fungus, and thus the rosacea, in the subject being treated.

In some embodiments, the method can further comprise topical administration of any one or any combination of corticosteroids to the subject. In some embodiments, the method can further comprise topical administration of any one or any combination of antibiotics to the subject. In some embodiments, the method can further comprise topical administration of any one or any combination of antibiotics and any one or any combination of corticosteriods to the subject.

Psoriasis is a condition that be treated using the methods taught herein. Psoriasis is considered to be a long-lasting autoimmune disease which is characterized by patches of abnormal skin, and these skin patches are typically red, itchy, and scaly, varying in severity from small and localized to complete body coverage. The main types of psoriasis can be treated using the methods provided herein and include plaque, guttate, inverse, pustular, and erythrodermic psoriasis. Plaque psoriasis, also known as psoriasis vulgaris, makes up about 90% of all cases and typically presents with red patches with white scales on top. Areas of the body most commonly affected are the back of the forearms, shins, around the navel, and the scalp. Guttate psoriasis has drop-shaped lesions. Pustular psoriasis presents with small non-infectious pus-filled blisters. Inverse psoriasis forms red patches in skin folds, and erythrodermic psoriasis occurs when the rash becomes very widespread, and can develop from any of the other types. One of skill in the art will appreciate that there is currently no cure for psoriasis. However, various treatments can help control the symptoms, and these treatments may include steroid creams, vitamin D3 cream, ultraviolet light, and immune system suppressing medications such as methotrexate. About 75% of cases can be managed with creams alone, and the disease typically affects 2-4% of the population.

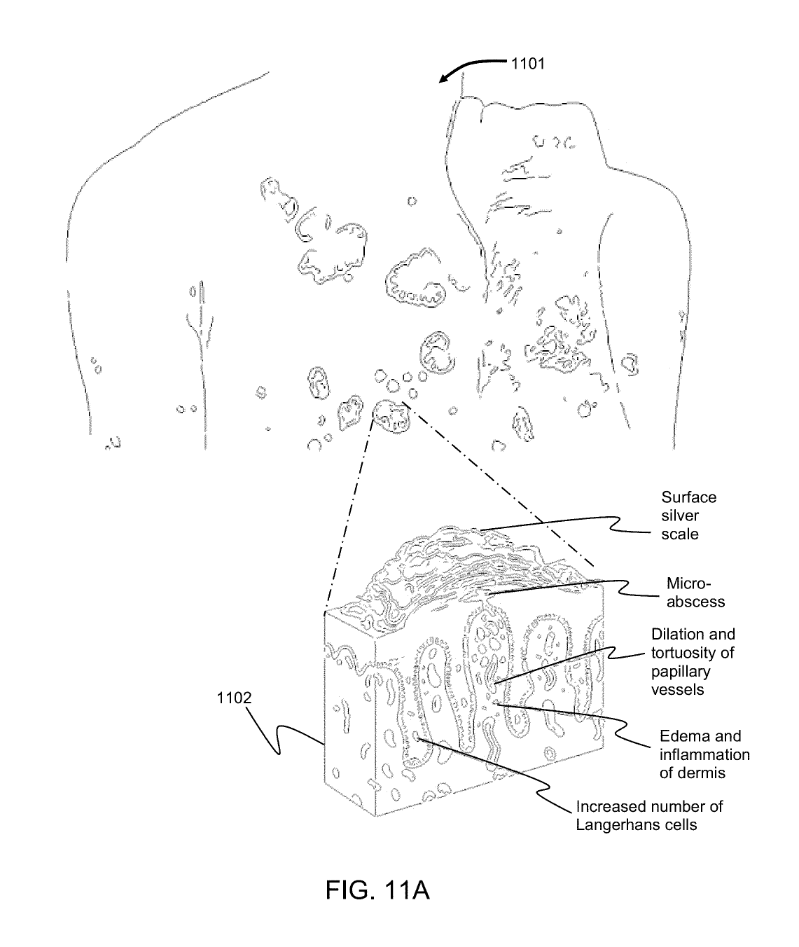

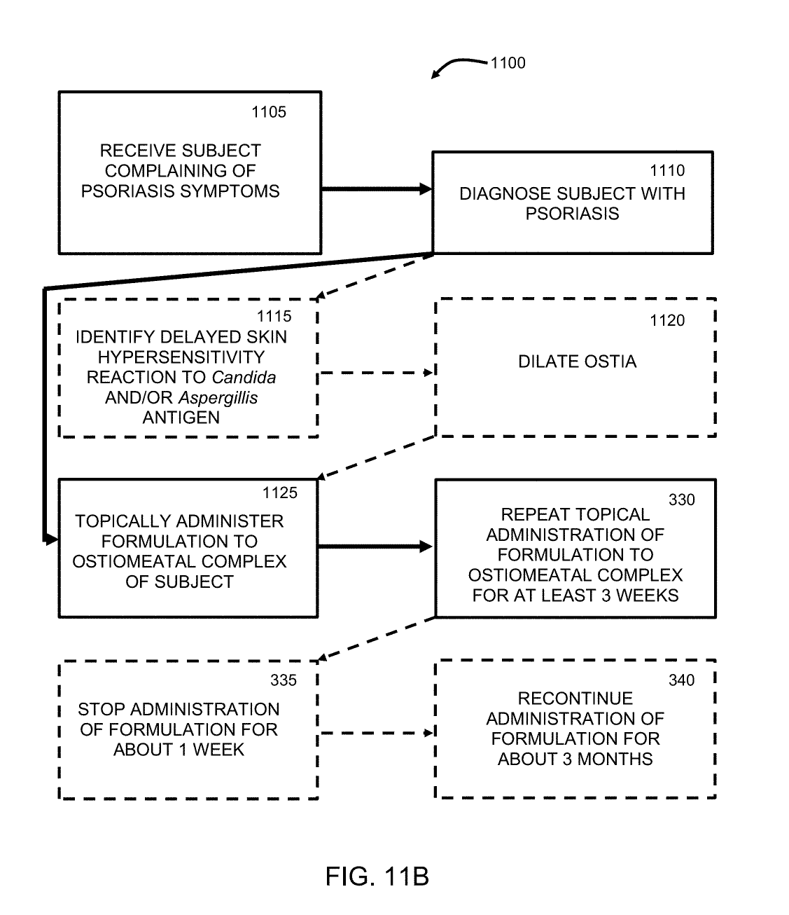

FIGS. 11A and 11B illustrate the use of the methods taught herein in the treatment of psoriasis, according to some embodiments. FIG. 11A illustrates a subject 1101 having psoriasis, as well as the tissue changes that occur on the skin of the subject 1101. The subject 1101 is suffering the deleterious effects of psoriasis that can include, for example, surface silver scale, micro abscesses, and dilation and tortuosity of papillary vessels.

In the method 1100 of FIG. 11B, such treatment methods can include first receiving 1105 a subject 1101 that is complaining of psoriasis symptoms and then diagnose 1110 the subject with psoriasis through methods that are well-known to the skilled artisan.

A topical administration of the combination of the antifungal agents can then be repeated 1130 and, in some embodiments, repeated for at least 3 weeks. In these embodiments, the topically administering can include contacting a first antifungal agent with the ostiomeatal complex, the first antifungal selected as effective at killing Candida species of fungus, inhibiting the growth and/or reproduction of the Candida species, or a combination thereof; and, contacting a second antifungal agent with the ostiomeatal complex, the second antifungal selected as effective at killing an Aspergillus species of fungus, inhibiting the growth and/or reproduction of the Aspergillus species, or a combination thereof. In such embodiments, the topically administering of the first antifungal agent can be concurrent with the topically administering of the second antifungal agent. It should be appreciated that the agents, compositions, and formulations taught herein are administered to effectively kill the survival spores of each type of fungus taught herein in order to at least inhibit, and preferably prevent, the recurrence of the fungus, and thus the psoriasis, in the subject being treated.

In some embodiments, the method can further comprise topical administration of any one or any combination of corticosteroids to the subject. In some embodiments, the method can further comprise topical administration of any one or any combination of antibiotics to the subject. In some embodiments, the method can further comprise topical administration of any one or any combination of antibiotics and any one or any combination of corticosteriods to the subject.

Cardiovascular disease is a condition that be treated using the methods taught herein. Cardiovascular disease involves the heart or blood vessels and includes coronary artery diseases (CAD) such as angina and myocardial infarction (commonly known as a heart attack). CVDs include stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis.

As such, as any cardiovascular disease can be treated using the methods and compositions provided herein, any cardiomyopathy can also be treated using the methods provided herein. Types of cardiomyopathy include hypertrophic cardiomyopathy, dilated cardiomyopathy, restrictive cardiomyopathy, arrhythmogenic right ventricular dysplasia, and broken heart syndrome. In hypertrophic cardiomyopathy, the heart muscle enlarges and thickens. In dilated cardiomyopathy the ventricles enlarge and weaken. In restrictive cardiomyopathy, the ventricle stiffens. Unfortunately, cardiomyopathy is difficult to treat, as the cause is generally unknown, such that the treatment is based on the type of cardiomyopathy and the degree of symptoms. Treatments currently are limited to lifestyle changes, medications, or surgery: (i) a healthy diet, including a variety of fruits, vegetables, and grains; choose foods low in saturated fat, trans fat, and cholesterol; prepare foods low in salt, and low in sugar; and, aim for a healthy weight; (ii) physical activity, and particularly with medical advice; (iii) quit smoking and avoid alcohol; (iv) get enough sleep and plenty of rest; (v) reduce stress; and, (vi) treat underlying conditions, such as diabetes, high blood pressure, and high cholesterol.

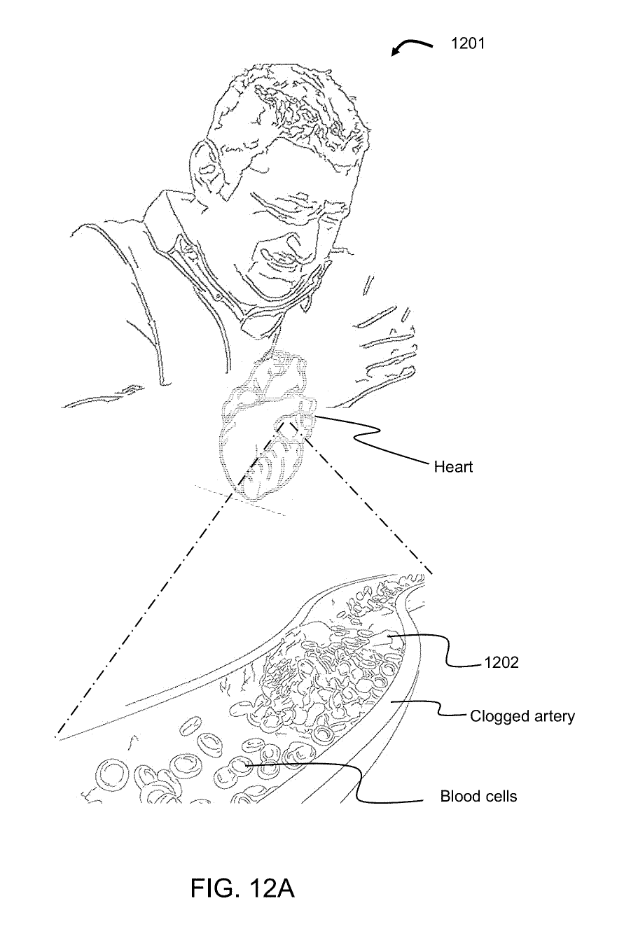

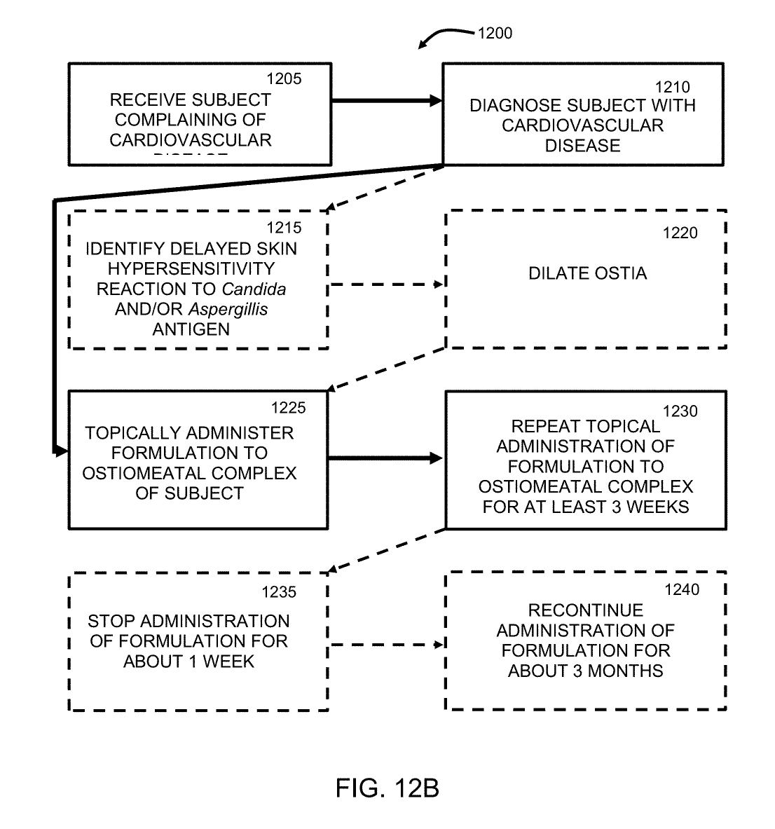

FIGS. 12A and 12B illustrate the use of the methods taught herein in the treatment of cardiovascular disease, according to some embodiments. FIG. 12A illustrates a subject 1201 having cardiovascular disease, as well as the tissue changes that occur on the skin of the subject 1201. The subject 1201 is suffering the deleterious effects of cardiovascular disease that can include, for example, arterial plaque, often from high cholesterol, and arterial blockage.

In the method 1200 of FIG. 12B, such treatment methods can include first receiving 1205 a subject 1201 that is complaining of cardiovascular disease symptoms and then diagnose 1210 the subject with cardiovascular disease through methods that are well-known to the skilled artisan.

A topical administration of the combination of the antifungal agents can then be repeated 1230 and, in some embodiments, repeated for at least 3 weeks. In these embodiments, the topically administering can include contacting a first antifungal agent with the ostiomeatal complex, the first antifungal selected as effective at killing Candida species of fungus, inhibiting the growth and/or reproduction of the Candida species, or a combination thereof; and, contacting a second antifungal agent with the ostiomeatal complex, the second antifungal selected as effective at killing an Aspergillus species of fungus, inhibiting the growth and/or reproduction of the Aspergillus species, or a combination thereof. In such embodiments, the topically administering of the first antifungal agent can be concurrent with the topically administering of the second antifungal agent. It should be appreciated that the agents, compositions, and formulations taught herein are administered to effectively kill the survival spores of each type of fungus taught herein in order to at least inhibit, and preferably prevent, the recurrence of the fungus, and thus the cardiovascular disease, in the subject being treated.

In some embodiments, the method can further comprise topical administration of any one or any combination of corticosteroids to the subject. In some embodiments, the method can further comprise topical administration of any one or any combination of antibiotics to the subject. In some embodiments, the method can further comprise topical administration of any one or any combination of antibiotics and any one or any combination of corticosteriods to the subject.

In any of the embodiments taught herein, the combination of the antifungal agents can be administered concurrently as a formulation. In some embodiments, the combination of antifungal agents can be administered in formulation with a corticosteroid and/or an antibiotic. In other words, in some embodiments, each of the antifungals can be administered separately or each administered as a component of the same formulation. Likewise, in some embodiments, the antibiotic can be administered separate from at least one of the antifungals and, in some embodiments, the antibiotic is administered with the combination of antifungals in the same formulation. Likewise, in some embodiments, the corticosteroid can be administered separate from at least one of the antifungals and, in some embodiments, the steroid can be administered with the combination of antifungals in the same formulation. It should be appreciated that the combination of antifungals, the antibiotic, and the steroid, can be administered together in the same formulation.

### Example 10. Method of Making a Formulation for Administration to a Target Tissue

As shown herein, it has been discovered that the use of a combination of antifungals provides an improved result over current methods of treatment. And, any combination of any antifungal taught herein can be used, in some embodiments. The antifungals can be combined in a ratio, for example, that can include a first antifungal, “Antifungal A” with one or more additional antifungals, which can be referred to as “Antifungal B.” Additional components can be added, as desired, for optimal efficacy. Unless otherwise taught or suggested, all weights of components provided herein are expressed as dry weights.

The following is an example composition:

TABLE 6

| | | AMOUNT PER |
|---|---|---|
| | AMOUNT | CAPSULE (mg); not |
| COMPONENT | MIXED (g) | adjusted for water |
| Antibiotic, e.g., tobramycin | 55.921 | 186 |
| sulfate (powder) | | |
| First antifungal, Antifungal A, | 12.006 | 40 |
| e.g., itraconazole | | |
| Second antifungal, Antifungal | 12.002 | 40 |
| B, e.g., ketoconazole. | | |
| Anti-inflammatory, e.g., | 0.180 | 0.6 |
| mometasone furoate | | |
| Excipient, xylitol 3% polox | 69.467 | 231 |
| base | | |

As such, one method of making a formulation for administration to a target tissue can include the following steps:

Identify Fungi of Interest.

One of skill will appreciate that the methods taught herein can be used to perform a skin test on the subject, if desired to identify the fungi of interest.

Select the Desired Antifungal Combination.

In some embodiments, Antifungal A can include any antifungal class or species taught herein, and Antifungal B can be include any one or any combination of antifungal classes or species taught herein. In some embodiments, Antifungal A is any antifungal that is effective at killing or at least inhibiting the growth of a Candida species of fungus, an Aspergillus species of fungus, or a combination thereof. In some embodiments, Antifungal B is any one or more antifungals that are effective at killing or at least inhibiting the growth of a Candida species of fungus, an Aspergillus species of fungus, or a combination thereof. For example, Antifungal A can be an imidazole class of antifungal or triazole class of antifungal, as taught herein, such that it can be selected from a group comprising itraconazole or ketoconazole, or both itraconazole and ketoconazole. And, likewise Antifungal B can include one or more imidazole or triazole classes of antifungals, as taught herein, such that it can be selected from a group comprising itraconazole or ketoconazole, or both itraconazole and ketoconazole. In some embodiments, the combination of Antifungal A and Antifungal B can include either itraconazole or ketoconazole, or both itraconazole and ketoconazole, as taught herein.

Select the Relative Amounts of Antifungals in the Combination.

In some embodiments, the ratios of Antifungal A to Antifungal B can range, for example, from 1:10⁶ to 10⁶ to 1, from 1:500×10³ to 500×10³:1, from 1:100×10³ to 100×10³:1, from 1:50×10³ to 10×50³:1, from 1:10×10³ to 10×10³:1, from 1:5000 or 5000:1, from 1:1000 to 1000:1, from 1:500 or 500:1, from 1:100 to 100:1, from 1:50 or 50:1, 1:10 to 10:1, 1:5 or 5:1, 1:4 or 4:1, 1:3 or 3:1, 1:2 or 2:1, 1:1, or any amount or range therein in increments of 0.1 per component in the ratio. In some embodiments, the ratio can be about 512:1, 256:1, about 128:1, about 64:1, about 32:1, about 16:1, about 8:1, about 4:1, about 2:1, or about 1:1 Antifungal A:Antifungal B, or any ratio therein in increments of 0.1 in the proportion of either component in the ratio. In some embodiments, the ratio can be about 512:1, 256:1, about 128:1, about 64:1, about 32:1, about 16:1, about 8:1, about 4:1, about 2:1, or about 1:1, or any ratio therein in increments of 0.1 in the proportion of either component in the ratio of imidazole:triazole or triazole:imidazole, and ketoconazole:itraconazole or itraconazole:ketoconazole.

Likewise, the amounts can be expressed in percent total antifungal, and, as such, in some embodiments there can be 0.1%, 0.5%, 1.0%, 2.0%, 3.0%, 4.0%, 5.0%, 10.0%, 20.0%, 30.0%, 40.0%, 50.0%, 60.0%, 70.0%, 80.0%, 90.0%, 95.0%, 96.0%, 97.0%, 98.0%, 99.0%, 99.1%, 99.5%, 99.6%, 99.7%, 99.8%, 99.9%, or any amount or range therein in increments of 0.1% of Antifungal A, which is combined with Antifungal B.

Select Additional Active Components, if any.

In some embodiments, the additional active components can include, for example, an antibiotic, an anti-inflammatory, or a combination thereof. The antibiotic and the anti-inflammatory should each be provided in at least “effective amounts”, as taught herein, amounts of which are known to those of skill in the art. In the example above, the tobramycin and mometasone furate were each incorporated in amounts that one of skill would believe to be an effective amount.

Select an Excipient.

In some embodiments, an excipient, if desired, can be one that one of skill will have reason to believe will function well in any respective embodiment taught herein. For example, in the formulation show above, xylitol was chosen as the excipient.

Select the delivery, such as aqueous administration, spray, droplet, nebulizer, etc, which includes selection of the volume of the carrier.

In some embodiments, the formulation can be administered in a volume of a liquid carrier, as taught herein. In some embodiments, the carrier can be an aqueous carrier ranging in volume from about 1 ml to about 50 ml, for example.

Table 7 provides the concentrations in mg/ml of each component shown in Table 6, given an aqueous carrier ranging in volume from 1-100 ml, in which a single capsule is dissolved in the aqueous carrier for a single administration, in some embodiments:

TABLE 7

| | | AMOUNT PER | |
|---|---|---|---|
| | | CAPSULE | |
| | | (mg); not | |
| | AMOUNT | adjusted for | CONCENTRATION PER |
| COMPONENT | MIXED (g) | water | CAPSULE (mg/ml) |
| Antibiotic, e.g., | 55.921 | 186 | 1.86 mg/ml to 186 mg/ml |
| tobramycin sulfate | | | |
| (powder) | | | |
| First antifungal, | 12.006 | 40 | 0.40 mg/ml to 40.0 mg/ml |
| Antifungal A, e.g., | | | |
| itraconazole | | | |
| Second antifungal, | 12.002 | 40 | 0.40 mg/ml to 40.0 mg/ml |
| Antifungal B, e.g., | | | |
| ketoconazole. | | | |
| Anti-inflammatory, e.g., | 0.180 | 0.6 | 0.006 mg/ml to 0.600 mg/ml |
| mometasone furoate | | | |
| Excipient, xylitol 3% | 69.467 | 231 | 2.31 mg/ml to 231 mg/ml |
| polox base | | | |

As shown in Table 7, the total antibiotic concentration ranges from about 1.86 mg/ml to about 186 mg/ml, at least for tobramycin. This, of course, can change depending on the selection of the antibiotic(s). In some embodiments, the antibiotic can be administered in concentrations ranging from about 0.1 mg/ml to about 1000 mg/ml, from about 0.1 mg/ml to about 500 mg/ml, from about 0.1 mg/ml to about 100 mg/ml, from about 1.0 mg/ml to about 500 mg/ml, from about 1.0 mg/ml to about 100 mg/ml, from about 10 mg/ml to about 500 mg/ml, from about 10 mg/ml to about 100 mg/ml, from about 100 mg/ml to about 500 mg/ml, or any range or amount therein in increments of 0.1 mg/ml. In some embodiments, the antibiotic can be administered in an amount of about 0.1 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10.0 mg/ml, 15.0 mg/ml, 20.0 mg/ml, 30.0 mg/ml, 40.0 mg/ml, 50.0 mg/ml, 60.0 mg/ml, 70.0 mg/ml, 80.0 mg/ml, 90.0 mg/ml, 100.0 mg/ml, 120.0 mg/ml, 140.0 mg/ml, 160.0 mg/ml, 180.0 mg/ml, 200.0 mg/ml, or any amount therein in increments of 0.01 mg/ml. In some embodiments, the antibiotic can be administered dry in an amount of about 0.1 mg, 1.0 mg, 2.0 mg, 3.0 mg, 4.0 mg, 5.0 mg, 6.0 mg, 7.0 mg, 8.0 mg, 9.0 mg, 10.0 mg, 15.0 mg, 20.0 mg, 30.0 mg, 40.0 mg, 50.0 mg, 60.0 mg, 70.0 mg, 80.0 mg, 90.0 mg, 100.0 mg, 120.0 mg, 140.0 mg, 160.0 mg, 180.0 mg, 200.0 mg, or any amount therein in increments of 0.1 mg/ml.

As shown in Table 7, the total antifungal concentration ranges from about 0.80 mg/ml to about 80 mg/ml, and the ratio of Antifungal A to Antifungal B is about 1:1, at least for itraconazole and ketoconazole. This, of course, can change depending on the selection of the antifungal combination. In some embodiments, the first antifungal, Antifungal A can be administered in concentrations ranging from about 0.01 mg/ml to about 1000 mg/ml, from about 0.1 mg/ml to about 500 mg/ml, from about 0.1 mg/ml to about 100 mg/ml, from about 1.0 mg/ml to about 500 mg/ml, from about 1.0 mg/ml to about 100 mg/ml, from about 10 mg/ml to about 500 mg/ml, from about 10 mg/ml to about 100 mg/ml, from about 100 mg/ml to about 500 mg/ml, or any range or amount therein in increments of 0.01 mg/ml. In some embodiments, the first antifungal, Antifungal A, can be administered in an amount of about 0.1 mg/ml, about 1.0 mg/ml, about 2.0 mg/ml, about 3.0 mg/ml, about 4.0 mg/ml, about 5.0 mg/ml, about 6.0 mg/ml, about 7.0 mg/ml, about 8.0 mg/ml, about 9.0 mg/ml, about 10.0 mg/ml, about 15.0 mg/ml, about 20.0 mg/ml, about 30.0 mg/ml, about 40.0 mg/ml, about 50.0 mg/ml, about 60.0 mg/ml, about 70.0 mg/ml, about 80.0 mg/ml, about 90.0 mg/ml, about 100.0 mg/ml, about 120.0 mg/ml, about 140.0 mg/ml, about 160.0 mg/ml, about 180.0 mg/ml, about 200.0 mg/ml, or any amount therein in increments of 0.01 mg/ml. In some embodiments, the first antifungal can be administered dry in an amount of about 0.1 mg, 1.0 mg, 2.0 mg, 3.0 mg, 4.0 mg, 5.0 mg, 6.0 mg, 7.0 mg, 8.0 mg, 9.0 mg, 10.0 mg, 15.0 mg, 20.0 mg, 30.0 mg, 40.0 mg, 50.0 mg, 60.0 mg, 70.0 mg, 80.0 mg, 90.0 mg, 100.0 mg, 120.0 mg, 140.0 mg, 160.0 mg, 180.0 mg, 200.0 mg, or any amount therein in increments of 0.1 mg/ml.

In some embodiments, the second antifungal, Antifungal B can be administered in concentrations ranging from about 0.1 mg/ml to about 1000 mg/ml, from about 0.1 mg/ml to about 500 mg/ml, from about 0.1 mg/ml to about 100 mg/ml, from about 1.0 mg/ml to about 500 mg/ml, from about 1.0 mg/ml to about 100 mg/ml, from about 10 mg/ml to about 500 mg/ml, from about 10 mg/ml to about 100 mg/ml, from about 100 mg/ml to about 500 mg/ml, or any range or amount therein in increments of 0.1 mg/ml. In some embodiments, the second antifungal, Antifungal B, can be administered in an amount of about 0.1 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10.0 mg/ml, 15.0 mg/ml, 20.0 mg/ml, 30.0 mg/ml, 40.0 mg/ml, 50.0 mg/ml, 60.0 mg/ml, 70.0 mg/ml, 80.0 mg/ml, 90.0 mg/ml, 100.0 mg/ml, 120.0 mg/ml, 140.0 mg/ml, 160.0 mg/ml, 180.0 mg/ml, 200.0 mg/ml, or any amount therein in increments of 0.1 mg/ml. In some embodiments, the second antifungal can be administered dry in an amount of about 0.1 mg, 1.0 mg, 2.0 mg, 3.0 mg, 4.0 mg, 5.0 mg, 6.0 mg, 7.0 mg, 8.0 mg, 9.0 mg, 10.0 mg, 15.0 mg, 20.0 mg, 30.0 mg, 40.0 mg, 50.0 mg, 60.0 mg, 70.0 mg, 80.0 mg, 90.0 mg, 100.0 mg, 120.0 mg, 140.0 mg, 160.0 mg, 180.0 mg, 200.0 mg, or any amount therein in increments of 0.1 mg/ml.

In some embodiments, the concentration of the total antifungal can range from about 0.01 mg/mL to about 1000 mg/mL, 0.01 mg/mL to 500 mg/mL, 0.01 mg/mL to 400 mg/mL, 0.01 mg/mL to 300 mg/mL, 0.01 mg/mL to 200 mg/mL, 0.01 mg/mL to 100 mg/mL, from about 0.01 mg/mL to about 1000 mg/mL, 0.01 mg/mL to 500 mg/mL, 0.01 mg/mL to 400 mg/mL, 0.02 mg/mL to 300 mg/mL, 0.02 mg/mL to 200 mg/mL, 0.02 mg/mL to 100 mg/mL, from about 0.03 mg/mL to about 1000 mg/mL, 0.03 mg/mL to 500 mg/mL, 0.03 mg/mL to 400 mg/mL, 0.03 mg/mL to 300 mg/mL, 0.03 mg/mL to 200 mg/mL, 0.03 mg/mL to 100 mg/mL, from about 0.05 mg/mL to about 1000 mg/mL, 0.05 mg/mL to 500 mg/mL, 0.05 mg/mL to 400 mg/mL, 0.05 mg/mL to 300 mg/mL, 0.05 mg/mL to 200 mg/mL, 0.05 mg/mL to 100 mg/mL, from about 0.5 mg/mL to about 1000 mg/mL, 0.5 mg/mL to 500 mg/mL, 0.5 mg/mL to 400 mg/mL, 0.5 mg/mL to 300 mg/mL, 0.5 mg/mL to 200 mg/mL, 0.5 mg/mL to 100 mg/mL, from about 1.0 mg/mL to about 1000 mg/mL, 1.0 mg/mL to 500 mg/mL, 1.0 mg/mL to 400 mg/mL, 1.0 mg/mL to 300 mg/mL, 1.0 mg/mL to 200 mg/mL, 1.0 mg/mL to 100 mg/mL, 2.0 mg/mL to 200 mg/mL, 3.0 mg/mL to 400 mg/mL, 4.0 mg/mL to 400 mg/mL, 5.0 mg/mL to 500 mg/mL, 10.0 mg/mL to 1000 mg/mL, from about 10.0 mg/mL to 500 mg/mL, from about 10.0 mg/mL to 250 mg/mL, 20.0 mg/mL to 200 mg/mL, 20.0 mg/mL to 400 mg/mL, 20.0 mg/mL to 400 mg/mL, 50.0 mg/mL to 500 mg/mL, 100 mg/mL to 1000 mg/mL, 100 mg/mL to 500 mg/mL, 100 mg/mL to 250 mg/mL, 100 mg/mL to 200 mg/mL, 100 mg/mL to 300 mg/mL, 100 mg/mL to 400 mg/mL, 100 mg/mL to 500 mg/mL, 100 mg/mL to 1000 mg/mL, or any amount or range therein in increments of 0.01 mg/ml. As such, the total antifungal can be administered in an amount of about 0.1 mg/ml, about 1.0 mg/ml, about 2.0 mg/ml, about 3.0 mg/ml, about 4.0 mg/ml, about 5.0 mg/ml, about 6.0 mg/ml, about 7.0 mg/ml, about 8.0 mg/ml, about 9.0 mg/ml, about 10.0 mg/ml, about 15.0 mg/ml, about 20.0 mg/ml, about 30.0 mg/ml, about 40.0 mg/ml, about 50.0 mg/ml, about 60.0 mg/ml, about 70.0 mg/ml, about 80.0 mg/ml, about 90.0 mg/ml, about 100.0 mg/ml, about 120.0 mg/ml, about 140.0 mg/ml, about 160.0 mg/ml, about 180.0 mg/ml, about 200.0 mg/ml, about 300 mg/ml, about 400 mg/ml, about 500 mg/ml, or any amount therein in increments of 0.01 mg/ml.

As shown in Table 7, the total antibiotic concentration ranges from about 0.006 mg/ml to about 0.600 mg/ml, at least for mometasone furate. This, of course, can change depending on the selection of the anti-inflammatory or anti-inflammatories. In some embodiments, the anti-inflammatory can be administered in concentrations ranging from about 0.1 mg/ml to about 1000 mg/ml, from about 0.1 mg/ml to about 500 mg/ml, from about 0.1 mg/ml to about 100 mg/ml, from about 1.0 mg/ml to about 500 mg/ml, from about 1.0 mg/ml to about 100 mg/ml, from about 10 mg/ml to about 500 mg/ml, from about 10 mg/ml to about 100 mg/ml, from about 100 mg/ml to about 500 mg/ml, or any range or amount therein in increments of 0.1 mg/ml. In some embodiments, the anti-inflammatory can be administered in an amount of about 0.1 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10.0 mg/ml, 15.0 mg/ml, 20.0 mg/ml, 30.0 mg/ml, 40.0 mg/ml, 50.0 mg/ml, 60.0 mg/ml, 70.0 mg/ml, 80.0 mg/ml, 90.0 mg/ml, 100.0 mg/ml, 120.0 mg/ml, 140.0 mg/ml, 160.0 mg/ml, 180.0 mg/ml, 200.0 mg/ml, or any amount therein in increments of 0.1 mg/ml. In some embodiments, the anti-inflammatory can be administered dry in an amount of about 0.1 mg, 1.0 mg, 2.0 mg, 3.0 mg, 4.0 mg, 5.0 mg, 6.0 mg, 7.0 mg, 8.0 mg, 9.0 mg, 10.0 mg, 15.0 mg, 20.0 mg, 30.0 mg, 40.0 mg, 50.0 mg, 60.0 mg, 70.0 mg, 80.0 mg, 90.0 mg, 100.0 mg, 120.0 mg, 140.0 mg, 160.0 mg, 180.0 mg, 200.0 mg, or any amount therein in increments of 0.1 mg/ml.

One of skill will appreciate that the methods and compositions disclosed herein, such as the treatment of each disorder discussed herein, for example, provide significant benefits compared with conventional treatment methods and compositions. Conventional methods and compositions typically address only one particular disease and/or fail to effectively treat the disease or inflammation. Given the ability of the presently disclosed methods and compositions to address the underlying autoimmune reaction responsible for inflammation in subjects, such methods and compositions may result in significant clinical benefits to subjects with, for example, antigen-mediated inflammation, particularly those with multiple diseases mediated by an autoimmune reaction to the same secretory antigen. Moreover, the methodologies and the various embodiments thereof described herein are exemplary. Various other embodiments of the methodologies described herein are possible. In addition, various publications are referenced herein, and the disclosures of these publications, in their entireties, are hereby incorporated by reference into this application to describe more fully the state of the art to which this application pertains. The references disclosed are also individually and specifically incorporated herein by reference for material contained within them that is discussed in the sentence in which the reference is relied on.

We claim:

1. A method of treating rosacea, the method comprising:
topically administering a combination of antifungal agents to a tissue infected with a combination of a Candida species of fungus and an Aspergillus species of fungus, the tissue in a subject having rosacea, and the topically administering including
contacting the tissue with a first antifungal agent, the first antifungal agent selected as effective at killing the Candida species, inhibiting the growth and/or reproduction of the Candida species, or a combination thereof; and,
contacting the tissue with a second antifungal agent, the second antifungal agent selected as effective at killing the Aspergillus species, inhibiting the growth and/or reproduction of the Aspergillus species, or a combination thereof;
wherein, the topically administering of the first antifungal agent is concurrent with the topically administering of the second antifungal agent, and the topically administering alleviates a symptom of rosacea in the subject.

2. The method of claim 1, wherein the tissue is the ostiomeatal complex.

3. The method of claim 1, wherein the combination of antifungals includes an imidazole.

4. The method of claim 8, wherein the imidazole is itraconazole.

5. The method of claim 1, wherein the combination of antifungals includes a triazole.

6. The method of claim 5, wherein the triazole is ketoconazole.

7. The method of claim 1, wherein the combination of antifungals includes an imidazole and a triazole.

8. The method of claim 7, wherein the combination of antifungals includes itraconazole and ketoconazole.

9. The method of claim 1, further comprising administering an antibiotic to the subject.

10. The method of claim 1, wherein the antibiotic is an aminoglycoside.

11. The method of claim 8, wherein the aminoglycoside is tobramycin.

12. The method of claim 10, wherein the antibiotic is a glycopeptide.

13. The method of claim 1, wherein the glycopeptide is vancomycin.

14. The method of claim 1, further comprising administering a corticosteroid to the subject.

15. The method of claim 14, wherein the corticosteroid is a Group III or Group IV corticosteroid selected from the group consisting of triamcinolone acetonide, mometasone furoate, fluticasone propionate, betamethasone dipropionate, halometasone, fluocinolone acetonide, hydrocortisone valerate, hydrocortisone butyrate, flurandrenolide, and triamcinolone acetonide.

16. The method of claim 14, wherein the corticosteroid is mometasone furoate.

17. The method of claim 1, wherein the combination of the antifungal agents are administered concurrently as a formulation with
a Group III or Group IV corticosteroid selected from the group consisting of triamcinolone acetonide, mometasone furoate, fluticasone propionate, betamethasone dipropionate, halometasone, fluocinolone acetonide, hydrocortisone valerate, hydrocortisone butyrate, flurandrenolide, and triamcinolone acetonide;
and,
an antibiotic selected from the group consisting of an aminoglycoside and a glycopeptide;
wherein, the administering is repeated daily for at least 3 weeks.

18. The method of claim 1, further comprising dilating sinus ostia, and the administering is repeated daily for at least 3 weeks.

19. The method of claim 1, wherein:
the method further comprises dilating sinus ostia;
the combination of the antifungal agents include itraconazole and ketoconazole administered concurrently as a formulation that includes
a corticosteroid that includes mometasone furoate; and,
an antibiotic that includes tobramycin and/or vancomycin;
wherein, the administering is repeated daily for at least 3 weeks, and then once a day for at least 1 additional month.

20. A formulation for the treatment of rosacea in a subject, the formulation comprising a first antifungal selected as effective at killing Candida species of fungus, inhibiting the growth and/or reproduction of the Candida species, or a combination thereof; and, a second antifungal selected as effective at killing an Aspergillus species of fungus, inhibiting the growth and/or reproduction of the Aspergillus species, or a combination thereof; wherein the formulation alleviates a symptom of rosacea in the subject.

21. The formulation of claim 25, wherein:
the first antifungal includes itraconazole;
the second antifungal includes ketoconazole;
and, the formulation further comprises a corticosteroid that includes mometasone furoate; and, an antibiotic that includes tobramycin and/or vancomycin;
wherein, the formulation at least substantially inhibits the recurrence of a fungal infection of the Candida species and the Aspergillus species in a group of subjects receiving an administration of the formulation to each of their respective ostiomeatal complexes when compared to a group of subjects having the fungal infection and not receiving an administration of the formulation.
